SEQUENCE LISTING (10 sequences)

SEQ ID NO: 1 - DNA (other nucleic acid), 30 bp; single-stranded, unknown
  description: /desc = “DNA Primer”
ggcataagcttgatatctgaagccatgggc

SEQ ID NO: 2 - DNA (other nucleic acid), 28 bp; single-stranded, unknown
  description: /desc = “DNA Primer”
gcgcggttaaccgttatcaggaaaatgc

SEQ ID NO: 3 - DNA (other nucleic acid), 39 bp; single-stranded, unknown
  description: /desc = “DNA Primer”
ggggatatccaccatggratgsagctgkgtmatsctctt

SEQ ID NO: 4 - DNA (other nucleic acid), 39 bp; single-stranded, unknown
  description: /desc = “DNA Primer”
gactgggtcatctggatgtcggagtggacacctgtggag

SEQ ID NO: 5 - DNA (other nucleic acid), 38 bp; single-stranded, unknown
  description: /desc = “DNA Primer”
ctccacaggtgtccactccgacatccagatgacccagt

SEQ ID NO: 6 - DNA (other nucleic acid), 32 bp; single-stranded, unknown
  description: /desc = “DNA Primer”
gcttgtcgacttacgtttgatctccaccttgg

SEQ ID NO: 7 - DNA (other nucleic acid), 39 bp; single-stranded, unknown
  description: /desc = “DNA Primer”
gactccaccagctgaacctcggagtggacacctgtggag

SEQ ID NO: 8 - DNA (other nucleic acid), 38 bp; single-stranded, unknown
  description: /desc = “DNA Primer”
ctccacaggtgtccactccgaggttcagctggtggagt

SEQ ID NO: 9 - DNA (other nucleic acid), 29 bp; single-stranded, unknown
  description: /desc = “DNA Primer”
ttggtgctagccgaggagacggtgaccag

SEQ ID NO: 10 - DNA (other nucleic acid), 27 bp; single-stranded, unknown
  description: /desc = “DNA Primer”
ggcggcggatccgaggttcagctggtg

(19) United States
(12) Patent Application Publication
(10) Pub. No.: US 2003/0171551 A1
(43) Pub. Date: Sep. 11, 2003

(54) CHIMERIC ANTIBODY FUSION PROTEINS FOR THE RECRUITMENT AND STIMULATION OF AN ANTITUMOR IMMUNE RESPONSE

(76) Inventors: JOSEPH D. ROSENBLATT, ROCHESTER, NY (US); PIA CHALLITA-EID, ROCHESTER, NY (US); SHERIE MORRISON, LOS ANGELES, CA (US); CAMILLE N. ABBOUD, ROCHESTER, NY (US); SEUNG-UON SHIN, LOS ANGELES, CA (US)

Correspondence Address: MICHAEL L GOLDMAN ESQ, NIXON PEABODY LLP, CLINTON SQUARE, P O BOX 1051, ROCHESTER, NY, 14603

(21) Appl. No.: 09/016,743

(22) Filed: Jan. 30, 1998

Related U.S. Application Data
(60) Provisional application No. 60/037,256, filed on Jan. 31, 1997.
(60) Provisional application No. 60/064,018, filed on Nov. 3, 1997.

(51) Int. Cl. A61K 39/395; A61K 39/40; A61K 39/42; C07K 1/00; C07K 14/00; C07K 17/00; C12P 21/08; C07K 16/00

(52) U.S. Cl. 530/388.8; 530/350; 530/387.3; 530/387.7; 424/133.1; 424/134.1; 424/138.1

(57) ABSTRACT

The present invention relates to chimeric molecules for the stimulation of an anti-tumor immune response to facilitate immune eradication of breast, ovarian and other cancer cells. The chimeric molecules include a binding region which specifically binds to a tumor specific antigen and a chemokine and/or costimulatory ligand. The invention further provides methods for inducing a tumor specific immune response and compositions which can be administered to mammals.

The present application claims the benefit of U.S. Provisional Patent Application Serial No. 60/037,256, filed Jan. 31, 1997, and 60/064,018, filed Nov. 3, 1997.

The present invention was developed under National Cancer Institute Grants Nos. EDT76502, CA16858, CA59326, and CA61472. The United States Government may have certain rights.

### FIELD OF THE INVENTION

The present invention relates to chimeric molecules having a binding domain which is specific for tumor associated antigens and a peptide having the activity of either a chemokine or a costimulatory ligand. Compositions providing the chimeric molecules either individually or together are also provided. The invention also relates to methods of treating tumor cells with one or more chimeric molecules and compositions for administration to a mammal.

### BACKGROUND OF THE INVENTION

The management of residual disease is a central problem in breast and other solid tumors. Despite efforts to maximize dose intensity, relapse remains a critical and generally fatal problem in high risk breast cancer patients. Chemotherapeutic strategies are necessarily limited by various toxicities, and of limited efficacy against nonproliferating tumor cells. Additional modalities, which will achieve further cytoreduction are needed. A variety of investigators have suggested the use of gene transfer techniques to augment immunogenicity of cancer cells, and provoke an immune tumor-directed response. Many of these strategies involve ex vivo manipulation of tumor cells, are technically difficult to implement, and do not target systemic tumor deposits.

Although various different trials of monoclonal antibodies, antibody based conjugates and/or radioantibody have been performed, with limited success, results of these trials have highlighted obstacles to successful antibody therapy of human malignancy. Antibody opsonization generally does not result in direct cytotoxicity, due to poor fixation of complement and/or poor enlistment of antibody dependent cytotoxicity (ADCC) (Junghans, R. P. et al., “Antibody-Based Immunotherapies for Cancer,” in Chabner et al., eds., Cancer Chemotherapy and Biotherapy, 2nd Ed., Philadelphia, Pa., 655-89 (1996); Schlom, J., “Antibodies in Cancer Therapy: Basic Principles of Monoclonal Antibodies,” in DeVita et al., eds., Biologic Therapy of Cancer, New York:J. B. Lippincott Co., 464-81 (1991)). Strategies based on direct antibody-based killing (e.g. antibody-toxin conjugates such as antibody-ricin, or radiolabeled antibody strategies, e.g. ¹³¹I-Ab) require delivery to all tumor cells and are hampered by limited vascular permeability to proteins of 150 kd or greater (mw of IgG) and extravascular diffusion ability (Jain, R. K., “Transport of Molecules Across Tumor Vasculature,” Cancer and Metastasis Reviews, 6:559-93 (1987); Jain, R. K., “Transport of Molecules in the Tumor Interstitium: A Review,” Cancer Res., 47:3039-51 (1987); Jain, R. K., “Determinants of Tumor Blood Flow: A Review,” Cancer Res., 48:2641-58 (1988); Jain, R. K., “Barriers to Drug Delivery in Solid Tumors,” Sci. Amer., 1:58-65 (1994)). Elevated interstitial pressures within tumor masses due to absent/poorly organized lymphatics further impede delivery. Antibody (Ab) and cytokine activation of effector cells may be more effective than Ab alone (LeBerthon, B. L. et al., “Enhanced Tumor Uptake of Macromolecules Induced by a Novel Vasoactive IL-2 Immunocongugate,” Cancer Res., 51:2694 (1991); Hank, J. A. et al., “Augmentation of ADCC Following In vivo Therapy with Recombinant IL-2,” Cancer Res., 50:5234-39 (1990)).

Stimulation of an antitumor immune response is a stepwise process requiring the accumulation and activation of immune effector cells in the vicinity of tumor cells. Monocytes and lymphocytes initially interact with adhesion molecules on endothelial cells, followed by migration of immune effector cells in response to chemotactic gradients in tissues. Effector cells in the tumor vicinity are then available for activation and subsequent stimulation of an antitumor immune response. Chemokines are low molecular weight proteins that act as potent chemoattractants, and are involved in migration of inflammatory cells. They are divided according to the configuration of the first cysteine residues at the amino terminus of the protein. Different subfamilies of chemokines have been shown to attract different classes of inflammatory cells. C-C chemokines predominantly attract monocytes and lymphocytes, while C-X-C chemokines attract neutrophils in addition to lymphocytes (For review, see, Mackay, C., “Chemokines: What Chemokine is That?” Curr Biol, 7:R384-6 (1997)). RANTES is a member of the C-C chemokine family and is a potent chemoattractant of T cells, NK cells, monocytes, eosinophils, basophils and dendritic cells (Taub, D., “Chemokine-Leukocyte Interactions. The Voodoo That They Do So Well,” Cytokine Growth Factor Rev, 7:355-76 (1996); Proost, P. et al., “The Role of Chemokines in Inflammation,” Int J Clin Lab Res, 26:211 (1996)). RANTES, present at high concentrations (1 μM), has also been shown to stimulate T cell activation and proliferation (Bacon, K., et al., “Activation of Dual T Cell Signaling Pathways by the Chemokine RANTES,” Science, 269:1727-1730 (1995); Taub, D., et al., “Chemokines and T Lymphocyte Activation: I. Beta Chemokines Costimulate Human T Lymphocyte Activation in Vitro,” J Immunol, 156:2095-2103 (1996). RANTES-mediated T cell activation can also lead to the generation of an antitumor immune response and tumor rejection as shown in gene transfer studies performed in murine syngeneic in vivo EL4 lymphoma (Mahmood, K.; Federoff, H.; Haltman, M.; Challita-Eid, P. M.; Rosenblatt, J. D., manuscript submitted) and MCA-205 tumor models (Mule, J., et al., “RANTES Secretion By Gene-Modified Tumor Cells Results in Loss of Tumorigenicity In Vivo: Role of Immune Cell Subpopulations,” Hum Gene Ther, 7:1545-1553 (1996). Therefore, direct delivery of RANTES to tumor deposits may assist in recruitment and/or the molecule may be used as a modulator for cancer immunotherapy.

T-cell activation and proliferation requires two signals from antigen-presenting cells (APCs). The first signal is antigen specific and mediated by recognition of antigenic peptides presented in the context of MHC-I or II by the T-cell receptor (TCR). A second or “costimulatory” signal can be provided via binding of a costimulatory ligand of the B7 family on the APC to the CD28 counterreceptor present on T-cells. The B7 family includes several Ig-like molecules including B7.1 and B7.2. Provision of signal 1 without signal 2 may lead to a state of immune tolerance (Guinan, E. et al., “Pivotal Role of the B7:CD28 Pathway in Transplantation Tolerance and Tumor Immunity,” Blood 84:3261-82 (1994)). B7.1 gene transfer into nonimmunogenic tumor cells has been shown to elicit a T-cell-mediated immune response not only against transfected (B7+) but also against parental nontransfected tumor cells (Chen, L. et al., “Costimulation of Antitumor Immunity by the B7 Counterreceptor for the T Lymphocyte Molecules CD28 and CTLA-4,” Cell 71:1093-102 (1992); Chen, L. et al., “Tumor Immunogenicity Determines the Effect of B7 Costimulation on T Cell-Mediated Tumor Immunity,” J Exp Med 179:523-32 (1994); Li, Y. et al., “Costimulation of Tumor-Reactive CD4+ and CD8+ T Lymphocytes by B7, a Natural Ligand for CD28, Can be Used to Treat Established Mouse Melanoma,” J. Immunol. 153:421-8 (1994); Dohring, C. et al., “T-Helper- and Accessory-Cell-Independent Cytotoxic Responses to Human Tumor Cells Transfected with a B7 Retroviral Vector,” Int J Cancer 57:754-9 (1994); Marti, W. et al., “Nonreplicating Recombinant Vaccinia Virus Encoding Human B-7 Molecules Elicits Effective Costimulation of Naive and Memory CD4+ T Lymphocytes in Vitro,” Cell Immunol 179:146-52 (1997); Hodge, J. et al., “Admixture of a Recombinant Vaccinia Virus Containing the Gene for the Costimulatory Molecule B7 and a Recombinant Vaccinia Virus Containing a Tumor-Associated Antigen Gene Results in Enhanced Specific T-Cell Responses and Antitumor Immunity,” Cancer Res 55:3598-603 (1995); Hodge, J. et al., “Induction of Antitumor Immunity by Recombinant Vaccinia Viruses Expressing B7-1 or B7-2 Costimulatory Molecules,” Cancer Res 54:5552-5 (1994)). Since T-cell activation requires both B7.1 activation and TCR engagement, only cells with TCRs which recognize antigenic determinants on tumor cells should be activated (Linsley, P. et al., “Binding of the B Cell Activation Antigen B7 to CD28 Costimulates T Cell Proliferation and Interleukin 2 mRNA Accumulation,” J Exp Med 173:721-30 (1991); Baskar, J., et al., “Constitutive Expression of B7 Restores Immunogenicity of Tumor Cells Expressing Truncated Major Histocompatibility Complex Class II Molecules,” Proc Natl Acad Sci USA 90:5687 (1993)).

Chemical conjugation of antibody to cytokines instead of fusion has resulted in decreased T-cell activation by the conjugate although effects on vascular permeability are preserved (Behr, T., et al., “Targeting of Liver Metastases of Colorectal Cancer with IgG, F(ab′)2, and Fab′ Anti-Carcinoembryonic Antigen Antibodies Labeled with 99mTc: The Role of Metabolism and Kinetics,” Cancer Res 55:5777s (1995)). In contrast, recent studies using an anti-tumor antibody-IL-2 fusion protein suggest retention of both antibody specificity and cytokine function in the fusion molecule (Gillies, S. D. et al., “Antibody-Targeted Interleukin-2 Stimulates T-cell Killing of Autologous Tumor Cells,” Proc. Natl. Acad. Sci., USA, 89:1428-32 (1992); Sabzevari, H. S. et al., “A Recombinant Antibody-Interleukin 2 Fusion Protein Suppresses Growth of Hepatic Human Neuroblastoma Metastases in Severe Combined Immunodeficiency Mice,” Proc. Natl. Acad. Sci. USA, 91:9626 (1994); Becker, J. C. et al., “Eradication of Human Hepatic and Pulmonary Melanoma Metastases in SCID Mice by Antibody-Interleukin 2 Fusion Proteins,” Proc. Natl. Acad. Sci. USA, 93:2702 (1996); Becker, J. C. et al., “An Antibody-Interleukin 2 Fusion Protein Overcomes Tumor Heterogeneity by Induction of a Cellular Immune Response,” Proc. Natl. Acad. Sci. USA, 93:7826 (1996); Becker, J. C. et al., “T Cell-Mediated Eradication of Murine Metastatic Melanoma Induced by Targeted Interleukin 2 Therapy,” J. Exp. Med., 183:2361 (1996); Harvill, E. T. et al., “An IgG3-IL-2 Fusion Protein Has Higher Affinity Than hrIL-2 for the IL-2R Alpha Subunit: Real Time Measurement of Ligand Binding,” Mol. Immunol., 33:1007 (1996); Reisfeld, R. A. et al., “Antibody-Interleukin 2 Fusion Proteins: A New Approach to Cancer Therapy,” J. Clin. Lab., 10:160 (1996); Harvill, E. T. et al., “In vivo Properties of an IgG3-IL-2 Fusion Protein. A General Strategy for Immune Potentiation,” J. Immunol., 157:3165 (1996)).

B7.1 gene transfer is not always a realistic option for treating cancer in a mammal. B7.1 gene transfer requires either ex vivo manipulation of tumor cells which is technically difficult, or in vivo delivery via gene therapy vectors which would not specifically target systemic tumor deposits. An effective method would not rely on absolute kill of all tumor cells by antibody/conjugate nor upon delivery to all tumor cells to elicit a response. The present invention overcomes the significant problems with biodistribution and delivery associated with prior methods.

### SUMMARY OF THE INVENTION

The present invention relates to a chimeric molecule having a binding domain capable of binding to a tumor cell associated antigen and a chemokine or an active fragment of a chemokine. The chimeric molecule is connected such that the binding domain remains capable of binding to the tumor cell associated antigen and the chemokine or fragment of the chemokine retains its activity.

The invention also relates to a method for stimulating a tumor specific immune response by providing a chimeric molecule, having a binding domain capable of binding to a tumor cell associated antigen and a chemokine or active fragment of a chemokine, and administering the fusion molecule to a mammal.

The invention further provides a chimeric molecule that has a binding domain capable of binding to a tumor cell associated antigen connected to a T-cell costimulatory ligand or to an active fragment of a costimulatory ligand. The chimeric molecule is connected such that the binding domain remains capable of binding to the tumor cell associated antigen and the costimulatory ligand or fragment of the costimulatory ligand retains its activity.

Another aspect of the invention is a method for stimulating a tumor specific immune response by providing a chimeric molecule. The chimeric molecule has a binding domain capable of binding to a tumor cell associated antigen and a costimulatory ligand or active fragment of a costimulatory ligand, and administering the fusion molecule to a mammal.

In addition to providing methods using chimeric molecules having either a chemokine or costimulatory ligand, the invention also provides a method for stimulating a tumor specific immune response where both chimeric molecules are administered to a mammal.

The invention also provides a composition for stimulating a tumor specific immune response having a chimeric molecule comprising a binding domain capable of binding to a tumor cell associated antigen and a chemokine or active fragment of a chemokine, a chimeric molecule comprising a binding domain capable of binding to a tumor cell associated antigen and a costimulatory ligand or active fragment of a costimulatory ligand, and a pharmaceutically-acceptable carrier.

In an alternative embodiment, the invention provides a chimeric molecule suitable for stimulating a tumor specific immune response having a binding domain capable of specifically binding to a tumor cell associated antigen and two or more T-cell effectors. The T-cell effectors can be a chemokine, a cytokine, or a costimulatory molecule or an active fragment of any of the proceeding. The T-cell effectors are associated with the binding domain such that the binding domain remains capable of binding the tumor cell associated antigen and the T-cell effectors retain activity.

### BRIEF DESCRIPTION OF THE DRAWINGS

FIG. 1 models the tumor specific activation of T-cells by Ig/B7.1 and Ig/RANTES fusions proteins. The anti-tumor/RANTES fusion protein attracts T cells into close proximity of the tumor site. The anti-tumor/B7.1 fusion protein acts on the T-cells stimulating proliferation and tumor specific cytotoxic activity.

FIG. 2 diagrams the construction of the antibody fusion constructs. The extracellular domain of RANTES or B7.1 obtained by polymerase chain reaction and the heavy chain variable region of the anti-tumor Ig are cloned on opposite ends of a flexible region. The resulting clone is cloned into a human IgG3 expression construct. The heavy chain IgG3 construct and a kappa light chain construct are transfected into Sp2/o Myeloma cells. The fusion protein is then secreted from the cells.

FIG. 3 depicts the vector construction for the expression of RANTES.Her2.IgG3. RANTES was cloned at the 5′ terminus of human IgG3 heavy chain through a flexible linker maintaining the open reading frame of the fusion protein. After transfection of both anti-HER2/neu light chain and RANTES heavy chain fusion genes into myeloma cells, an H₂L₂ form of the anti and secreted.

FIG. 4 is an SDS-PAGE analysis of the secreted recombinant antibodies. (A) Myeloma cells secreting her2.IgG3 (lanes 1 and 3) or RANTES.Her2.IgG3 (lane 2 and 4) were metabolically-labelled with ³⁵S-methionine, the supernatant was precipitated with goat anti-human IgG followed by Staph A, electrophoresed on an SDS-PAGE gel in absence (lanes 1 & 2) or presence (lanes 3 & 4) of 2-mercaptoethanol (2-ME), and analyzed by autoradiography. Her2.IgG3 (Lane 1) or RANTES.Her2.IgG3 (Lane 2) purified from culture supernatants were run on an SDS-PAGE gel, blotted onto nitrocellulose membrane and analyzed using HRP-conjugated anti-human Ig (B), or mouse anti-RANTES followed by HRP-conjugated anti-mouse antibody (C). The western blots were developed using a chemiluminescent substrate and analyzed by exposure to X-ray film.

FIG. 5 is the flow cytometry analysis of the recombinant antibodies. SKBR3 cells were incubated with either an isotype control antibody (a and d), Her2.IgG3 (b and e) or RANTES.Her2.IgG3 (c and f), as described in Materials and Methods, washed and stained with either FITC-conjugated anti-human IgG (a, b and c) or biotin-conjugated anti-RANTES antibody followed by PE-conjugated streptavidin (d, e and f). (g) EL4 or (h) EL4/HER2 cells were incubated with RANTES.Her2.IgG3, washed, stained with FITC-conjugated anti-human IgG. The samples were then analyzed by flow cytometry.

FIG. 6 shows the results of affinity studies of IgG3 and RANTES.Her2.IgG3 proteins to their antigen. Binding of IgG3 or RANTES.Her2.IgG3 to ECD coated microcuvette was assayed using an IAsys Optical Biosensor system as described in Materials and Methods and the association (kₐ) and dissociation (k_{d}) constants calculated using the Fastfit program. The affinity constant K_{D} was calculated as K_{d}/Kₐ. Binding following the addition of both proteins at 1×10⁻⁷ M is shown.

FIG. 7 shows F-actin polymerization of differentiated THP-1 cells. THP-1 were prestimulated with cAMP, washed and incubated with either rRANTES, RANTES.Her2.IgG3 or IgG3. At different time intervals, an aliquot of the cells was fixed with paraformaldehyde and stained with NBD-phallacidin. The samples were analyzed by flow cytometry and relative F-actin was calculated as mean fluorescence relative to time 0. This experiment was repeated three times with similar results.

FIG. 8 summarizes the transendothelial migration of peripheral blood T cells in response to soluble RANTES.Her2.IgG3. The average of the migration indexes for all four experiments described in Table 1 is plotted. The error bars represent standard error mean (SEM).

FIG. 9 shows the transendothelial migration of primary peripheral blood T cells in response to cell surface antigen-bound RANTES.Her2.IgG3. SKBR3 cells were preincubated with Her2.IgG3 or RANTES.Her2.IgG3 for 2 hours at 4° C. The SKBR3 cells were then washed and placed in the lower well of a transwell plate in which a confluent HUVEC monolayer was grown on the porous membrane. In separate wells, rRANTES was added at the indicated concentrations instead of preincubated SKBR3 cells. Purified peripheral blood T cells, at 3×10⁵ cells per well, were added to the upper well and the transwell plates were incubated at 37° C. overnight. Migration was measured by counting the number of T cells in the lower well. Background migration in presence of medium only was subtracted from the sample cell number.

FIG. 10 provides the structure of her2.IgG3 and B7.her2.IgG3 molecules. The heavy and light chain variable regions of humanized humAb4D5 anti-HER2/neu were cloned between the EcoRV sites and NheI sites of the mammalian expression vector for human IgG3 previously described (Coloma, M. et al., “Novel Vectors for the Expression of Antibody Molecules Using Variable Regions Generated by Polymerase Chain Reaction,” J Immunol Methods 152:89-104 (1992), which is hereby incorporated by reference). For the construction of B7.her2.IgG3, the B7.1 leader and extracellular domain were joined to the (Ser-Gly₄)₃ linker sequences which had been fused to the amino terminus heavy chain variable sequences of the her2.IgG3 antibody. A schematic diagram of the secreted H₂L₂ forms of control her2.IgG3 and B7.her2.IgG3 are also shown.

FIG. 11 is an SDS-PAGE analysis of the recombinant anti-HER2/neu antibodies. Cell lines expressing her2.IgG3 (lanes 1 and 3) or B7.her2.IgG3 (lanes 2 and 4) were labelled by overnight growth in medium containing ³⁵S-methionine. Supernatants from labelled cells were immunoprecipitated with goat anti-human IgG and protein A, and precipitated proteins analyzed by SDS-PAGE in the absence (lanes 1 and 2) or presence (lanes 3 and 4) of 2-mercaptoethanol.

FIG. 12 shows the results of flow cytometry to detect binding of her2.IgG3 of B7.her2.IqG3 to cell-surface expressed HER2/neu antigen. Parental CHO (A & D) or her2 expressing CHO/Her2 cells (B, C, E & F) were incubated with 10 μg/ml of either her2.IgG3(A, B & C) or B7.her2.IgG3 (D, E & F) at 4° C. for 2 hours. The cells were washed and stained with either FITC-conjugated anti-human IgG (A, B, D & E) or PE-conjugated anti-human B7.1 (C&F) at 4° C. for 30 minutes. The cells were then analyzed by flow cytometry.

FIG. 13 shows the affinity of her2.IgG3 and B7.her2.IgG3 for HER2/neu determined using the IASYS biosensor. Binding of her2.IgG3 or B7.her2.IgGe to HER2/neu ECD coated microcuvette was assayed as described in Experimental Protocol and the k_{d}/kₐ.

FIG. 14 demonstrates binding of B7.1 to its counter-receptors CD28 and CTLA4 determined by slot blot (A) or flow cytometry assays (B). (A) 100 or 20 ng of CTLA4Ig or CD28Ig immobilized on a nitrocellulose membrane was incubated with either purified her2.IgG3 or B7.her2.IgG3 followed by alkaline phosphatase-conjugated anti-human kappa. The blots were then developed with BCIP/NBT substrate. (B) Parental CHO (a and c) or CHO/CD28 cells (b and d) were incubated with either soluble human B7.1 in the form of B7Ig) (a and b) or with B7.her2.IgG3 fusion protein (c and d). The cells were then washed, incubated with FITC-labelled anti-human IgG and analyzed by flow cytometry.

FIG. 15 shows the stability of recombinant anti-HER2/neu antibodies on the surface of antigen-expressing breast cancer cells. SKBR3 cells were incubated at 4° C. with either anti-her2.IgG3 (panels a and c) or B7.her2.IgG3 (panels b and d) and the amount of antibody bound determined by immunofluorescence. The cells were washed, incubated at 37° C. and aliquots removed at 0,1,3 or 24 hours, stained with FITC-conjugated ant IgG and analyzed by flow cytometry. In (A) flow cytometry results are shown at time 0 (a & b) and 24 hours after incubated at 37° C. (c & d). In (b) mean fluorescence is calculated as a percentage of the maximum mean fluorescence observed at time 0. The experiment was repeated three times with similar results.

FIG. 16 is an In vitro T-cell proliferation assay. Peripheral blood T-cells isolated from blood of normal donors A and B were plated in 96-well plates in presence of irradiated CHO and CHO/Her2 cells, PMA (10 ng/ml) and increasing concentrations of either her2.IgG3 or B7.her2.IgG3. The cocultures were incubated at 37° C. for 3 days and labelled with ³H-thymidine for the final 16-18 hours. (A) Proliferation was measured by harvesting the cells onto glass filters and assessing radioactivity by liquid scintillation counting. The results shown represent the average of triplicate cultures and error bars denote the standard error of the mean. Results from two separate experiments using two separate donors, donor A and donor B are shown. The experiment was repeated five times using a total of three different T-cell donors incubated in the presence of: (a) CHO/Her2 cells and 10 μg/ml her2.IgG3; (b) CHO/Her2 and 10 μg/ml B7.herIgG3; (c) CHO cells and 10 μg/ml B7.her2.IgG3; (d) CHO/Her2 in absence of antibody; or (e) CHO/B7 cells stably expressing human B7.1 by gene transfer.

FIG. 17 is a photograph of the cocultures showing the presence of proliferating T-cell colonies which are directly correlated with levels of proliferation detected by ³H-thymidine incorporation.

FIG. 18 provides tumor growth kinetics of EL4 cells (A) and MC38 cells (B), parental and transduced with human her2neu cDNA in vivo. Parental tumor cells or cells transduced with her2neu cDNA (10⁷ or 10⁵ cells) were injected s.c. either in the right or left flank of the leg of C57B1/6 mice respectively. Tumor growth was monitored using a caliper until the size reached about 20 mm in diameter at which time the mice were sacrificed.

FIG 19 shows the expression of her2/neu on MC38 cells following implantation in mice. Mice were injected in the right flank with 10⁶ MC38/Her2 bright cells. Two weeks later, one mouse was sacrificed, the tumor was dissected and dispersed in culture into single cell suspension. In FIG. 8A, the cells were then stained with control mouse IgG antibody or 4D5 mouse anti-her2/neu antibody, followed by FITC-labelled goat anti-mouse IgG. In FIG. 8B, the her2/neu positive bright population of MC38/her2 was sorted and expanded in culture.

### DETAILED DESCRIPTION OF THE INVENTION

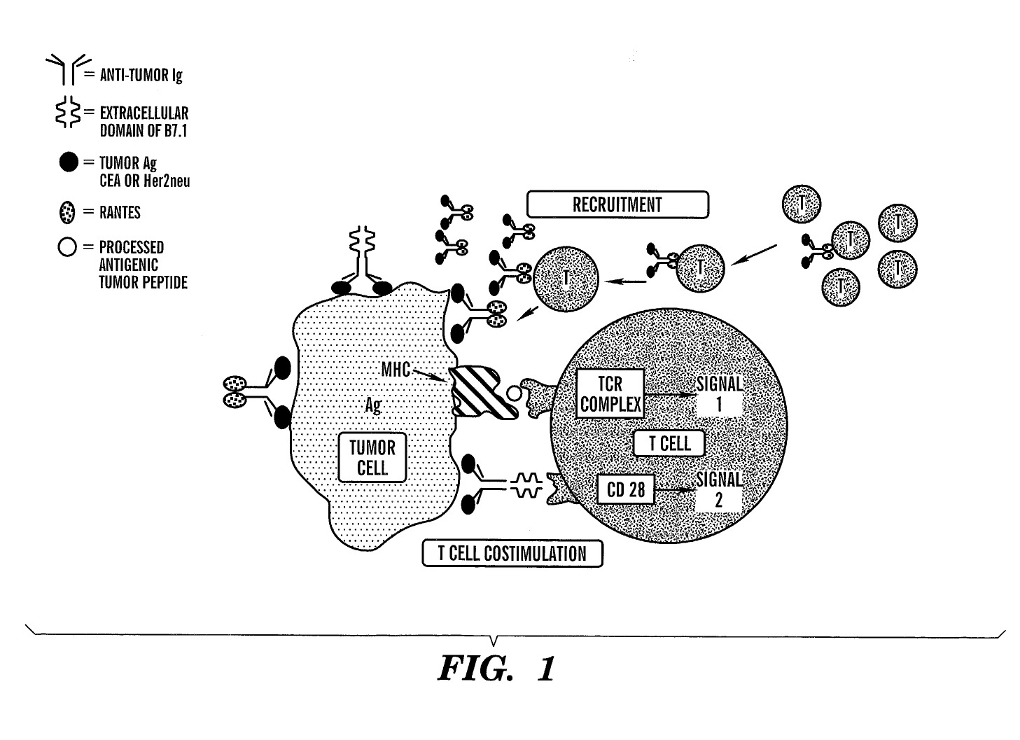

The present invention provides a novel approach for the stimulation of an anti-tumor immune response using chimeric molecules to facilitate immune eradication of breast, ovarian and other cancer cells. In particular, the present invention provides chimeric molecules directed against known tumor associated antigens e.g., Her2/neu and CEA, connected to the chemokine RANTES, or to the extracellular domain of the T-cell costimulatory ligand B7.1. (See FIG. 1)

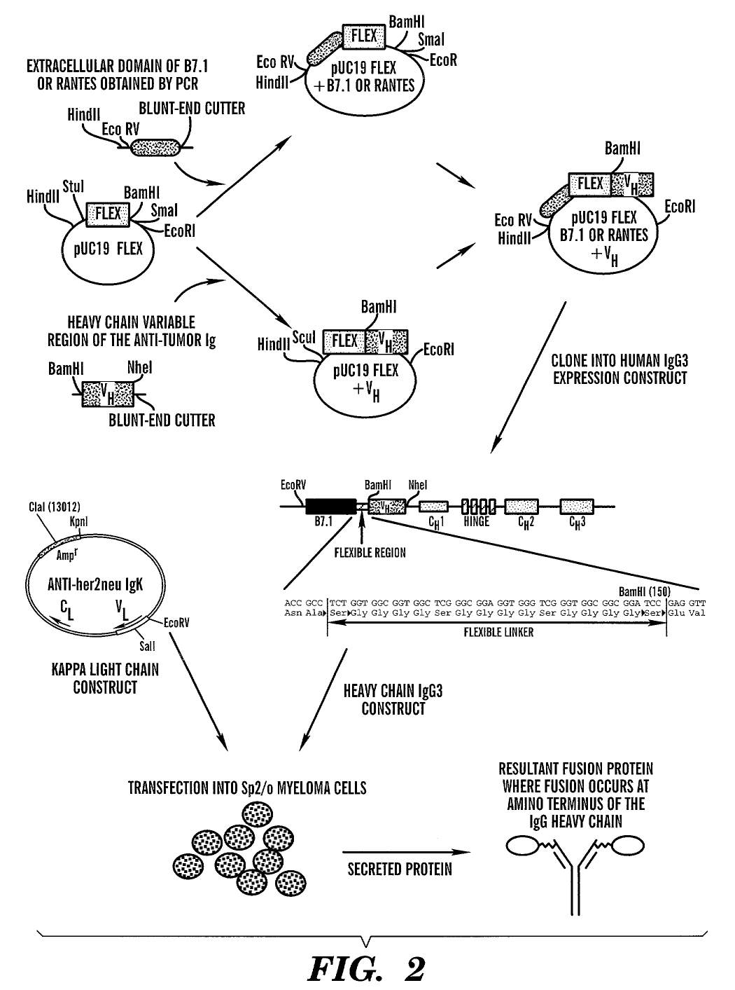

One aspect of the present invention relates to a chimeric molecule having a binding domain capable of binding to a tumor cell associated antigen and a chemokine or active fragment of a chemokine. The binding domain and the chemokine are connected such that the binding domain remains capable of binding to the tumor cell associated antigen and the chemokine retains activity. In a preferred embodiment of the invention, the chimeric molecule also has a flexible linker or hinge region located between the chemokine and the binding domain (See FIG. 2).

Preferred chemokines include DC-CK1, SDF-1, fractalkine, lymphotactin, IP-10, Mig, MCAF, MIP-1α, MIP-1β, IL-8, NAP-2, PF-4, and RANTES or an active fragment thereof. A more preferred embodiment is where the chemokine is RANTES.

Local tumor specific cellular immune responses require the availability of dendritic cells or other antigen presenting cells able to prime specific T cell effector responses (Linehan, D.C., et al., “Tumor-Specific and HLA-A2-Restricted Cytolysis by Tumor-Associated Lymphocytes in Human Metastatic Breast Cancer,” J Immunol 155:4486 (1995), which is hereby incorporated by reference). A novel class of low molecular weight factors, designated “chemokines” which elicit effector and/or antigen presenting cell migration to areas of inflammation has recently been characterized (Taub, D., “Chemokine-Leukocyte Interactions. The Voodoo That They Do So Well,” Cytokine Growth Factor Rev, 7:355-76 (1996); Proost, P. et al., “The Role of Chemokines in Inflammation,” Int J Clin Lab Res, 26:211 (1996); Murphy, W. J., et al., “Human RANTES Induces the Migration of Human T Lymphocytes into the Peripheral Tissues of Mice with Severe Combined Immune Deficiency,” Eur J Immunol 24:1823 (1994); Schall, T. J., et al., “Chemokines, Leukocyte Trafficking, and Inflammation,” Curr Opinion Immunol 6:865 (1994); Sozzani, S., et al., J Immunol 155:3292 (1995); Taub, D. D., et al., “Alpha and Beta Chemokines Induce NK Cell Migration and Enhance NK-Mediated Cytolysis,” J Immunol 155:3877 (1995), which are hereby incorporated by reference). RANTES, a member of the C-C family of chemokines is a potent chemoattractant of monocytes and basophils as well as of unstimulated CD4⁺/CD45RO⁺ memory T cells (Schall T. J., et al., “Selective Attraction of Monocytes and T Lymphocytes of the Memory Phenotype by Cytokine RANTES,” Nature 347:669 (1990), which is hereby incorporated by reference). Human RANTES is capable of inducing the release of granule enzymes from primary natural killer cells as well as cloned CTL lines, suggesting the involvement of RANTES in lymphocyte-dependent cytotoxicity as well as chemotaxis (Loetscher, P., et al., “Activation of NK Cells by CC Chemokines: Chemotaxis, Ca⁺⁺ Mobilization, and Enzyme Release,” J Immunol 156:322 (1996), which is hereby incorporated by reference). Chemokine lymphotactin in combination with either IL-2 or GM-CSF causes tumor cell infiltration with CD4⁺ and CD8⁺ T-cells, and provides increased protection from growth of preexisting tumors (Dilloo, D. et al., “Combined Chemokine and Cytokine Gene Trnaser Enhances Antitumor Immunity,” Nature Medicine, 2:1090 (1996), which is hereby incorporated by reference). RANTES has the additional potential advantage of causing direct T-cell proliferation when present at high concentrations (Bacon, K. B. et al., “Activation of Dual T Cell Signaling Pathways by the Chemokine RANTES,” Science, 269:1727 (1995), which is hereby incorporated by reference.

Local delivery of chemokines augments the potential efficacy of B7.1 delivery. RANTES, a low m.w. (8 kD) CC chemokine coupled to antibody coding sequences can be used specifically to recruit effector cells/APC's to the site of tumors (Sozzani, S., et al., J Immunol 155:3292 (1995); Taub, D. D., et al., “Alpha and Beta Chemokines Induce NK Cell Migration and Enhance NK-Mediated Cytolysis,” J Immunol 155:3877 (1995), which are hereby incorporated by reference). By coupling RANTES to a targeting antibody, the transendothelial migration of effector cells such as CD4⁺ T-lymphocytes, natural killer cells, and antigen presenting dendritic cells, is stimulated, thereby enhancing the T-cell repertoire and evoking an active cellular immune effector response in the tumor vicinity (Maass, G., et al., “Priming of Tumor-Specific T Cells in the Draining Lymph Nodes after Immunization with Interleukin 2-Secreting Tumor Cells: Three Consecutive Stages May be Required for Successful Tumor Vaccination,” Proc Natl Acad Sci USA 92:5540 (1995); Maghahazaki, A. A., et al., “C-C Chemokines Induce the Chemotaxis of NK and IL-2 Activated NK Cells. Role for G-proteins,” J Immunol 153:4969 (1994); Murphy, W. J., et al., “Human RANTES Induces the Migration of Human T Lymphocytes into the Peripheral Tissues of Mice with Severe Combined Immune Deficiency,” Eur J Immunol 24:1823 (1994); Schall, T. J., et al., “Chemokines, Leukocyte Trafficking, and Inflammation,” Curr Opinion Immunol 6:865 (1994); Sozzani, S., et al., J Immunol 155:3292 (1995); Taub, D. D., et al., “Alpha and Beta Chemokines Induce NK Cell Migration and Enhance NK-Mediated Cytolysis,” J Immunol 155:3877 (1995), which are hereby incorporated by reference).

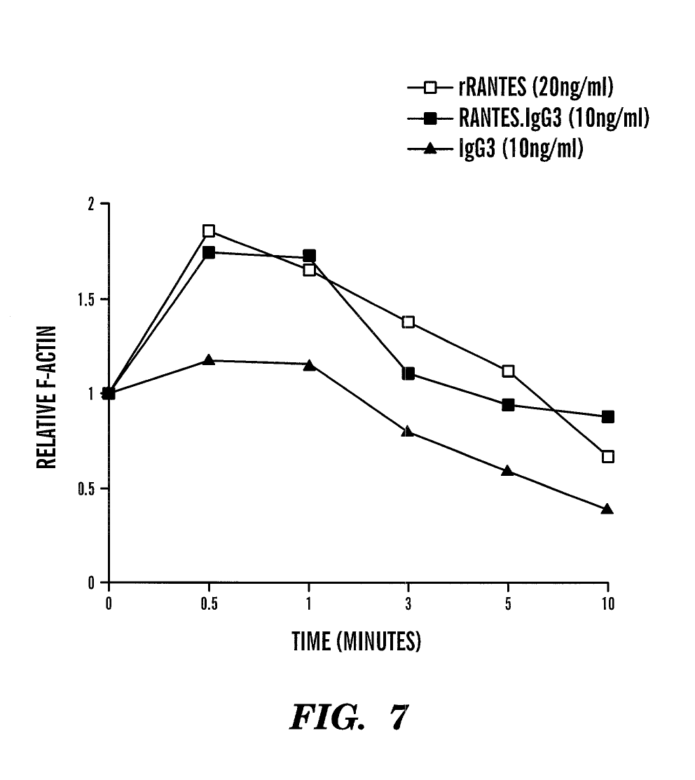

The biological activity of RANTES containing fusion proteins is determined by isolating peripheral blood mononuclear and T-cells from heparinized venous blood of normal volunteers. T-cell subsets and CO34+ precursor cells are purified using R&D affinity columns as published (de Waal Malefyt R., et al. “Interleukin 10 (IL-10) and Viral IL-10 Strongly Reduce Antigen-Specific Human T Cell Proliferation by Diminishing the Antigen-Presenting Capacity of Monocytes Via Downregulation of Class II Major Histocompatibility Complex Expression,” J Exp Med, 174:915 (1991); Winslow, J. M., et al., “CD34+ Progenitor Cell Isolation from Blood and Marrow: A Comparison of Techniques for Small Scale Selection,” Bone Marrow Transplant, 14:265 (1994), which are hereby incorporated by reference). Dendritic cells will be purified as published and analyzed by flow cytometry (McClellan, A. D., et al., “Isolation of Human Blood Dendritic Cells by Discontinuous Nycodenz Gradient Centrifugation,” J Immunol Methods, 184:81 (1995); Peshwa, M. V., et al., “Isolation of Peripheral Blood Dendritic Cells for Adoptive Cell Therapy,” Blood, 1:720 (1995), which are hereby incorporated by reference). These cells are then tested for chemotaxis when exposed to RANTES fusion proteins (FIG. 7).

In a preferred embodiment of the invention, the chimeric molecule has a binding domain which specifically binds to a tumor cell associated antigen from tumor cells which are breast cancer cells, ovarian cancer cells, lung cancer cells, prostate cancer cells, or other her2/neu expressing cancer cells.

The HER2/neu oncogene has been found to be amplified (>5+ copies) and/or overexpressed in as many as 30% of human breast, 10-30% of ovarian cancers, and a subset of lung and other cancers (Slamon, D. et al., “Human Breast Cancer: Correlation of Relapse and Survival with Amplification of the HER-2/neu Oncogene,” Science 235:177-82 (1987); Slamon, D., “Proto-Oncogenes and Human Cancers” [Editorial], N Enql J Med 317:955-7 (1987); Bacus, S. et al., “HER-2/neu Oncogene Expression and DNA Ploidy Analysis in Breast Cancer,” Arch Pathol Lab Med 114:164-9 (1990); Natali, P. et al., “Expression of the p185 Encoded by HER2 Oncogene in Normal and Transformed Human Tissues,” Int J Cancer 45:457-61 (1990), which are hereby incorporated by reference). Humanized anti-HER2/neu antibody has been demonstrated to be an effective therapeutic agent in several Phase I and II clinical trials (Pegram, M. et al., “Phase II Study of Intravenous Recombinant Humanized Anti-p185 HER-2 Monoclonal Antibody (rhuMAb HER-2) Plus Cisplatin in Patients with HER2/neu Overexpressing Metastatic Breast Cancer,” Proceedings of ASCO 14:106 (1995); Baselga, J. et al., “Phase II Study of Weekly Intravenous Recombinant Humanized Anti-p185HER2 Monoclonal Antibody in Patients with HER2/neu-Overexpressing Metastatic Breast Cancer [See Comments],” J Clin Oncol 14:737-44, which are hereby incorporated by reference). A Phase II trial showed response in over 11% of patients, and has led to ongoing Phase III trials (Baselga, J. et al., “Phase II Study of Weekly Intravenous Recombinant Humanized Anti-p185HER2 Monoclonal Antibody in Patients with HER2/neu-Overexpressing Metastatic Breast Cancer [See Comments],” J Clin Oncol 14:737-44, which is hereby incorporated by reference). These studies demonstrate the feasibility of targeting metastatic breast cancer through the HER2/neu antigen.

Carcinoembryonic antigen (“CEA”) is a valuable tumor marker used in the postoperative surveillance of tumors of epithelial origin such as colon, lung and breast and their metastases. CEA is a 180 kDa glycoprotein and belongs to the immunoglobulin superfamily. Elevated serum levels of CEA are associated with advanced breast cancer and CEA levels at least partially reflect disease progression (Kuroki, M., et al., “Serologic Mapping and Biochemical Characterization of the Carcinoembryonic Antigen Epitopes Using Fourteen Distinct Monoclonal Antibodies,” Int. J. Cancer 44:208 (1989); Mughal, A. W., et al., “Serial Plasma Carcinoembryonic Antigen Measurement During Treatment of Metastatic Breast Cancer,” JAMA 249:1881 (1983); Von Kleist, S., et al., “Immunodiagnosis of Tumors,” Eur. J. Cancer 29A:1622 (1993), which are hereby incorporated by reference). Quantitative autoradiographic analysis has shown CEA to be expressed in 83% of breast cancer specimens (Chung, J. K., et al., “Tumor Concentration and Distribution of Carcinoembryonic Antigen Measured by in vitro Quantitative Autoradiography,” J. Nuclear Med. 35:1499 (1994), which is hereby incorporated by reference).

Other types of tumor associated antigens may also be targeted, for example: EGF-R in bladder and breast cancer, prostate specific membrane antigen in prostate cancer, GD2 in neuroblastoma, membrane immunoglobulins in lymphomas, and/or T-cell receptors in T-cell lymphoma (LeMaistre, C. F. et al., “Targeting the EGF Receptor in Breast Cancer Treatmant, Breast Cancer Res Treat, 32:97 (1994); Israeli, R. S., et al., “Prostate-Specific Membrane Antigen and Other Prostatic Tumor Markers on the Horizon,” Urol Clin North Am, 24:439 (1997); Zhang, S. C. et al., “Selection of Tumor Antigens as Targets for Immune Attack Using Immunohistochemistry: I. Focus on Gangliosides,” Int J Cancer, 73:42 (1997); Mennel, H. D., et al., “Expression of GD2-Epitopes in Human Intracranial Tumors and Normal Brain,” Exp Toxicol Pathol, 44:317 (1992); Hoon, D. S. et al., “Aberrant Expression of Gangliosides in Human Renal Cell Carcinomas,” J Urol, 150:2013 (1993); Andrews, P. W., et al., “Comparative Analysis of Cell Surface Antigens Expressed by Cell Lines Derived from Human Germ Cell Tumors,” Int J Cancer, 66:806 (1996); Chang, H. R. et al., “Expression of Disialogangliosides GD2 and GD3 on Human Soft Tissue Sarcomas,” Cancer 70:633 (1992), which are hereby incorporated by reference).

Preferred fusion proteins are partially based on the 4D5 antibody successfully employed in Phase I/II trials. Overexpression of her2/neu in breast and ovarian cancers has also been shown to be associated with poor prognosis (Toikkanen, S., et al., “Prognostic Significance of Her2 Oncoprotein Expression in Breast Cancer: A 30-year Follow-up,” J Clin Oncol 8:103-112 (1992), which is hereby incorporated by reference). Hence, a proposed therapy would be specifically applicable to a high risk subset of patients. The her2/neu antigen is issued as a targeting mechanism for localization of B7.1 and RANTES to the tumor surface rather than as the primary antigen. The advantage of this approach is that while expression of her2/neu or CEA may be heterogeneous, targeting via her2/neu or CEA may activate T cells with specificity against other unidentified antigens, resulting in destruction of both her2/neu positive and nonexpressing cells.

The chemokine is preferentially fused to the amino terminus of either the heavy or light chain of an antibody molecule. A more preferred embodiment is where the chemokine is fused to the amino acid terminus of the heavy chain.

In addition to genetic techniques for forming fusion proteins, chimeric proteins may be created using other coupling methods. For example, the T-Cell effector molecule may be fused with an antibody, that binds to a tumor cell associated antigen, via avidin or strepavidin. Avidin or strepavidin conjugated to or directly fused to the antibody. (Edward A. Bayer et al, “The Avidin-Biotin Complex in Affinity Cytochemistry”, in Methods in Enzymology, Vol. 62 (1979), which is hereby incorporated by reference) Alternatively, chemical conjugation could be used to form a chimeric molecule.

The chimeric molecule preferably binds to a tumor cell associated antigen which is a cell surface antigen. In advanced disease, detectable levels of CEA are secreted into the circulation, which might affect antibody-based therapies. High expression of her2/neu in some patients might also lead to shedding of a secreted form of the antigen called ECD. Shedding can be accounted for by measurement of either circulating CEA and/or her2neu/ECD. Despite circulating CEA levels, monoclonal antibodies have been successfully used to localize colorectal and other tumors which express CEA (Behr, T., et al., “Targeting of Liver Metastases of Colorectal Cancer with IgG, F(ab′)2, and Fab′ Anti-Carcinoembryonic Antigen Antibodies Labeled with 99mTc: The Role of Metabolism and Kinetics,” Cancer Res 55:5777s (1995); Juhl, H., et al., “A Monoclonal Antibody-Cobra Venom Factor Conjugate Increases the Tumor-Specific Uptake of a 99mTc-Labeled Anti-Carcinoembryonic Antigen Antibody by a Two-step Approach,” Cancer Res 55:5749s (1995); Juweid, M., et al., “Targeting and Initial Radioimmunotherapy of Medullary Thyroid Carcinoma with 131I-Labeled Monoclonal Antibodies to Carcinoembryonic Antigen,” Cancer Res 55:5946s (1995); Sharkey, R. M., et al., “Evaluation of a Complementarity-Determining Region-Grafted (Humanized) Anti-Carcinoembryonic Antigen Monoclonal Antibody in Preclinical and Clinical Studies,” Cancer Res 55:5935s (1995); Wong, J. Y., et al., “Initial Experience Evaluating ⁹⁰yttrium-Radiolabeled Anti-Carcinoembryonic Antigen Chimeric T84.66 in a Phase I Radioimmunotherapy Trial,” Cancer Res 55:5929s (1995), which are hereby incorporated by reference). The Col-6 MAb specific for CEA does not react with either NCA and normal fecal antigen-1 making it a promising candidate for in vivo use (Kuroki, M., et al., “Serologic Mapping and Biochemical Characterization of the Carcinoembryonic Antigen Epitopes Using Fourteen Distinct Monoclonal Antibodies,” Int. J. Cancer 44:208 (1989); Robbins, P. F., et al., “Definition of the Expression of the Human Carcinoembryonic Antigen and Non-specific Cross-reacting Antigen in Human Breast and Lung Carcinomas,” Int. J. Cancer 53:892-7 (1993), which are hereby incorporated by reference). Although initial principles can be established using CEA and/or her2/neu as target antigen(s), future chimeric proteins could be derived against other tumor associated antigens or used in a minimal residual disease setting following surgery and/or adjuvant radiotherapy/chemotherapy in which TAA shedding into the circulation is less of a problem.

Antibodies used to bind selectively the products of the mutated genes can be produced by any suitable technique. For example, monoclonal antibodies may be produced in a hybridoma cell line according to the techniques of Kohler and Milstein, Nature, 265, 495 (1975), which is hereby incorporated by reference. A hybridoma is an immortalized cell line which is capable of secreting a specific monoclonal antibody. Purified polypeptides may be produced by recombinant means to express a biologically active isoform, or even an immunogenic fragment thereof may be used as an immunogen. Monoclonal Fab fragments may be produced in Escherichia coli from the known sequences by recombinant techniques known to those skilled in the art. (See, e.g., Huse, W., Science 246, 1275 (1989), which is hereby incorporated by reference) (recombinant Fab techniques).

The term “antibodies” as used herein refers to various types of immunoglobulin, including IgG, IgM, and IgA, and their relevant subclasses. The antibodies may be monoclonal or polyclonal and may be of any species of origin, including (for example) mouse, rat, rabbit, horse, or human, or may be chimeric antibodies, and include antibody fragments such as, for example, Fab, F(ab′)₂, and Fv fragments, and the corresponding fragments obtained from antibodies other than IgG.

In a preferred method, transfectomas can be grown in roller bottles or in a CellMax hollow fiber system for the large scale production of recombinant antibodies. Most of the transfectomas can be grown in low serum or serum-free medium. Binding to a Protein G (Gamma Bind columns from Pharmacia) has been shown to be a rapid and effective means of isolating recombinant proteins. Standard tools for protein purification, including an FPLC with ion exchange and sizing columns can also be used.

Purified protein may be obtained by several methods. The protein or polypeptide of the present invention is preferably produced in purified form (preferably at least about 80%, more preferably 90%, pure) by conventional techniques. Typically, the protein or polypeptide of the present invention is secreted into the growth medium of recombinant host cells. Alternatively, the protein or polypeptide of the present invention is produced but not secreted into growth medium. In such cases, to isolate the protein, the host cell carrying a recombinant plasmid is propagated, lysed by sonication, heat, chemical treatment, and the homogenate is centrifuged to remove cell debris. The supernatant is then subjected to sequential ammonium sulfate precipitation. The fraction containing the polypeptide or protein of the present invention is subjected to gel filtration in an appropriately sized dextran or polyacrylamide column to separate the proteins. If necessary, the protein fraction may be further purified by HPLC.

To evaluate the biologic activities and properties of the recombinant antibody fusion proteins in vitro, binding of fusion proteins to MC38 or EL4 expressing the target antigens (her2/neu or CEA) or other antigens is assessed by flow cytometry. The cloned anti-her2/neu variable region retains its specificity for her2/neu following fusion gene expression.

The molecular weight, the structural assembly between heavy and light chains, and the glycosylation pattern can be determined in the presence and absence of tunicamycin as performed for the anti DNS-B7.1 and IL-2 fusion proteins. Correct translation and folding of the B7.1 domain can be assessed using several methods. ELISA assays could be performed using a monoclonal antibody to B7.1, one such antibody is the BB-1 antibody to B7.1. Specificity and affinity of B7.1 binding to the costimulatory receptor(s) CTLA4 and CD28 is characterized by quantitative radioimmunoprecipitation with soluble CTLA4Ig and CD28Ig (CTLA4Ig from P. Linsley, and CD28Ig from Bristol-Meyers Squibb). CHO cell lines stably expressing either CD28 or B7.1 have been obtained from Dr. P. Linsley (Bristol-Meyers, Washington) (Linsley, P. S. et al., “Binding of the B Cell Activation Antigen B7 to CD28 Costimulates T Cell Proliferation and Interleukin 2 mRNA Accumulation,” J Exp Med, 173:721 (1991), which is hereby incorporated by reference). CD28+CHO cells are used to measure binding of the B7.1 antibody fusion proteins by flow cytometry using FITC-labelled anti-human IgG.

Human peripheral blood lymphocytes (“PBLs”) or Jurkat cells are stimulated in vitro in the presence of suboptimal concentrations of anti-CD3 (“OKT3”), or following stimulation with phorbol myristyl acetate (PMA), with and without immobilized antibody B7.1 fusion proteins, B7Ig or anti-CD28 (as positive controls), and T-Cell proliferative response assessed by ³H-thymidine incorporation. T-cell activation can be assessed further by measurement of IL-2 secretion/cytokine elaboration from cells in bulk or individually by flow cytometry and/or immunostaining.

Additionally, the fusion proteins may stimulate a Mixed-Leukocyte-Tumor-Reaction. In order to test for stimulation, fresh PBLs are incubated with irradiated tumor cells in the presence or absence of anti her2/neu-Ig/B7.1 fusion proteins. Ovarian carcinoma cell line OVCAR-3 (ATCC), which stimulates a T-Cell response when transfected with the B7.1 cDNA is used (Döhring, C., et al., “T-helper Accessory-Cell-Independent Cytotoxic Responses to Human Tumor Cells Transfected with a B7 Retroviral Vector,” Int J Cancer, 57:754 (1995), which is hereby incorporated by reference).

FcγR binding by the chimeric molecule can also be characterized. The chimeric molecules which are produced also have a Fc region attached and this Fc may provide an additional means of recruiting immune effector cells. Whether the chimeric molecules are capable of interacting with any of the FcRγ can be determined. To study FcγR, a binding competition assay is used.

Chimeric molecule interactions with complement may also affect the efficiency of the chimeric molecule in inducing an immune response. The relative efficiency of the recombinant fusion proteins in activating the complement cascade can also be determined. Many complement assays (direct lysis, consumption, C1q binding and C1 activation by Western blot) are routine and have been used to characterize recombinant proteins.

Preferred chimeric molecules would not be degraded rapidly in the mammal. Properties of the recombinant proteins in vitro and their effectiveness in causing tumor regression, such as in vivo half-life, can be determined by labelling purified recombinant proteins with ¹²⁵I, injecting it into normal mice, and then determining the half-life by whole body counting of the mice. A NaI detector with attached scaler with a well large enough to accommodate a mouse can be used. Animals would be sacrificed at different times after injection and isolated organs (e.g., brain, liver, lung, spleen) are counted. An alternate approach is to label the proteins biosynthetically with ³⁵S-methionine and determine serum half-life by serially sampling small quantities of blood obtained by eye or tail bleeding. To determine the maximum tolerated dose, mice are injected with increasing concentrations of the different proteins. The recipient mice are monitored for morbidity, weight gain and mortality. Techniques are also available for localization of the molecules in SCID mice (Park, G. W., et al., “Development of Anti-p185^{her2} Immunoliposomes for Cancer Therapy,” Proc. Nat'l Acad. Sci. USA 92:1327-31 (1995), which is hereby incorporated by reference).

The invention also provides a gene encoding the chimeric molecule. For example, one method of making the chimeric molecules is depicted in FIG. 2, in particular B7.1 and RANTES antibody fusion proteins specific for CEA and her2/neu. For each construct, the tumor specific fusion protein (e.g., anti-CEA/B7.1 or anti-CEA/RANTES), tumor specific antibodies lacking the fusion protein, and non-specific antibodies of the same structure can be compared. Vectors for the expression of antibodies recognizing CEA and her2/neu are produced. Plasmids are also produced encoding variable regions for “humanized” 4D5 her2/neu specific antibody from Dr. Paul Carter of Genentech, (Carter, P., et al., “Humanization of an Anti-p185HER2 Antibody for Human Cancer Therapy,” Proc Natl Acad Sci USA 89:4285 (1992), which is hereby incorporated by reference). PCR is used to modify the variable regions to make them suitable for use in the vectors. If needed, variable regions from additional hybridomas specific for her2/neu (ATCC) can be cloned. (Coloma, J. J., et al., “Novel Vectors for the Expression of Antibody Molecules Using Variable Regions Generated by PCR”, Immunol. Methods, 152:89 (1992), which is hereby incorporated by reference). Variable regions so cloned are expressed as fusion protein using the expression vectors. Several versions of the B7.1 and RANTES antibody fusion proteins can be constructed. Initially, three different antibody fusion proteins are being studied. In a first version, B7.1 or RANTES is fused to the carboxy-terminus of the Ig heavy chain. In a second version, they are fused to the amino-terminus of IgG3 via a flexible linker to make the amino-terminus of either RANTES or B7.1 more available for ligand binding, since it has been shown to be crucial for the activity of both B7.1 (Guo, Y., et al., “Mutational Analysis and an Alternatively Spliced Product of B7 Defines its D28/CTLA4-Binding Site in Immunoglobulin C-like Domain” J Exp Med 181:1345 (1995), which is hereby incorporated by reference) and RANTES (Wells, T. N. C., et al. “Peptides from the Amino-Terminus of RANTES Cause Chemotaxis of Human T-Lymphocytes” Biochem Biophy Res Com 211:100 (1995), which is hereby incorporated by reference), while preserving the antigen binding site. In a third version, they are fused to the carboxy-terminus, but with the inclusion of a flexible linker at the fusion site in order to provide more flexibility to the fusion.

The hB7.pBJ plasmid encoding the extracellular domain of B7.1 was obtained from Dr. L. Lanier (DNAX, California). Several of the B7.1 expression vectors needed have already been constructed and are discussed in the examples. The coding sequences are amplified by PCR, and then cloned into the appropriate vectors.

The expression vectors are transfected into host cells for expression. Transfection vectors, such as those developed by Oi and Morrison (Qi, VT, et al. “Chimeric Antibodies” BioTechniques (1986), which is hereby incorporated by reference), can be used in conjunction with the fusion protein cloning cassettes for expression of both IgG3 H and L chains. Electroporation is the preferred method for introducing DNA into host cells, for example myeloma cells (P3X63.Ag8.653, Sp2/0 or CHO). Stable transfectomas are isolated using the selectable drug markers and culture supernatant is screened by ELISA. Cytoplasmic and secreted chimeric proteins are labeled with ³⁵S-methionine, immunoprecipitated and analyzed by SDS-PAGE under reducing and non-reducing conditions to verify expected molecular weight.

U.S. Pat. No. 4,237,224 to Cohen and Boyer, which is hereby incorporated by reference, describes the production of other expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

Recombinant genes may also be introduced into viruses, such as adenovirus or herpes virus. Such viruses may be either defective or compentent for replication. Recombinant viruses can be generated by transfection of plasmids into cells infected with virus.

Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference.

A variety of host-vector systems may be utilized to express the protein-encoding sequence(s). Preferred vectors include a viral vector, plasmid, cosmid or an oligonucleotide. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eucaryotic promoters differ from those of procaryotic promoters. Furthermore, eucaryotic promoters and accompanying genetic signals may not be recognized in or may not function in a procaryotic system, and, further, procaryotic promoters are not recognized and do not function in eucaryotic cells.

Similarly, translation of mRNA depends upon the presence of the proper signals. Efficient translation of mRNA requires a ribosome binding site. This sequence is a short nucleotide sequence of mRNA that is located before the start codon, usually AUG, which encodes the amino-terminal methionine of the protein. The ribosome binding sites are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference.

A preferred embodiment of the invention is where the gene is functionally linked to a promoter. Promoters vary in their “strength” (i.e. their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in procaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

The present invention further provides host cells carrying the gene encoding the chimeric protein. Once the isolated DNA molecule encoding the human origin of recognition complex polypeptide or protein has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, mammalian cells, insect cells, plant cells, and the like.

The invention further provides a method for stimulating a tumor specific immune response. In one embodiment, a nucleic acid molecule encoding a chimeric molecule comprising a binding domain capable of binding to a tumor cell associated antigen and a chemokine is introduced into cells of the mammal and the gene is then expressed in the mammal.

Alternatively, a chimeric molecule comprising a binding domain capable of binding to a tumor cell associated antigen and a chemokine or active fragment of a chemokine is directly administered to a mammal. Preferred mammals include rats, mice, and humans. In particular, humans are the preferred mammals.

The chimeric molecule may be administered to the mammal orally, intradermally, intramuscularly, intrapleurally, intraperitoneally, intravenously, subcutaneously, or intranasally.

Alternatively, a nucleic acid molecule which can express the chimeric protein can be introduced into the mammal to produce the chimeric protein. The nucleic acid molecule can be delivered to the tumor, to circulating immune cells, or to human fibroblasts for expressing the chimeric protein at the site of the tumor.

The nucleic acid molecule may be introduced into the target cells using delivery vehicles capable of delivering the chimeric protein into the cells of the mammal (Dow et al., U.S. Pat. No. 5,705,151 (1998), which is hereby incorporated by reference). The target site may be a cancer cell, a tumor, or a lesion caused by an infectious agent, or an area around such cell, tumor or lesion, which is targeted by direct injection or delivery using liposomes or other delivery vehicles. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in an animal, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a cancer cell can be introduced to the outer surface of a liposome delivery vehicle so as to target the delivery vehicle to the cancer cell. Tumor cell ligands include ligands capable of binding to a molecule on the surface of a tumor cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Another preferred delivery vehicle comprises a recombinant virus particle vaccine. A recombinant virus particle vaccine of the present invention includes a therapeutic composition of the present invention, in which the recombinant molecules contained in the composition are packaged in a viral coat that allows entrance of DNA into a cell so that the DNA is expressed in the cell. A number of recombinant virus particles can be used, including, but not limited to, those based on alphaviruses, poxviruses, adenoviruses, herpesviruses, arena virus and retroviruses.

Another preferred delivery vehicle comprises a recombinant cell vaccine. Preferred recombinant cell vaccines of the present invention include tumor vaccines, in which allogeneic (i.e., cells derived from a source other than a patient, but that are histiotype compatible with the patient) or autologous (i.e., cells isolated from a patient) tumor cells are transfected with recombinant molecules contained in a therapeutic composition, irradiated and administered to a patient by, for example, intradermal, intravenous or subcutaneous injection. Therapeutic compositions to be administered by tumor cell vaccine, include recombinant molecules of the present invention without carrier. Tumor cell vaccine treatment is useful for the treatment of both tumor and metastatic cancer. Use of a tumor vaccine of the present invention is particular useful for treating metastatic cancer, including preventing metastatic disease, as well as, curing existing metastatic disease.

Yet another embodiment of the invention is a composition for stimulating a tumor specific immune response having the chimeric molecule with a chemokine connected to a tumor specific antibody and a pharmaceutically-acceptable carrier.

Another embodiment of the invention is chimeric molecules having a binding domain capable of binding to a tumor cell associated antigen and a costimulatory ligand or active fragment thereof. The binding domain and costimulatory ligand are connected such that the binding domain remains capable of binding to the tumor cell associated antigen and the costimulatory ligand retains activity.

Another aspect of the present invention relates to a chimeric molecule having a binding domain capable of binding to a tumor cell associated antigen and a costimulatory ligand or active fragment of a costimulatory ligand. The binding domain and the costimulatory ligand are connected such that the binding domain remains capable of binding to the tumor cell associated antigen and the costimulatory ligand retains activity. In a preferred embodiment of the invention, the chimeric molecule also has a flexible linker or hinge region located between the chemokine and the binding domain (See FIG. 2).

The costimulatory ligand is preferentially a B7.1 or B7.2. The preferred costimulatory ligand is B7.1. Targeting of B7.1 to the tumor cell surface, using an antibody-B7.1 fusion, can specifically stimulate T-cell clones with affinity for determinants presented in the context of MHC-I/II.

T-cell activation and function require two signals from antigen-presenting cells (“APCs”). The first signal is antigen specific and mediated by recognition by the T-cell receptor (“TCR”) of antigenic peptides in the context of MHC-I or MHC-II molecules. A second signal is provided by costimulation via binding of the B7.1 molecule expressed on APCs to CD28 and/or CTLA4 present on activated T-cells (Allison, J. P., et al., “The Yin and Yang of T cell Costimulation,” Science 270:932 (1995); Baskar, S., et al., “Constitutive Expression of B7 Restores Immunogenicity of Tumor Cells Expressing Truncated Major Histocompatibility Complex Class II Molecules,” Proc Natl Acad Sci USA 90:5687 (1993); Chen, L., et al., “Tumor Immunogenicity Determine the Effect of B7 Costimulation on T Cell-Mediated Tumor Immunity,” J Exp Med 179:523 (1994); Chen, L., et al., “Costimulation of Antitumor Immunity by the B7 Counterreceptor for the T Lymphocyte Molecules CD28 and CTLA-4,” Cell 71:1093 (1992); Guinan, E. C., et al., “Pivotal Role of the B7:CD28 Pathway in Transplantation Tolerance and Tumor Immunity,” Blood 84:3261 (1994); Linsley, P. S., et al., “Binding of the B Cell Activation Antigen B7 to CD28 Costimulates T Cell Proliferation and Interleukin 2 mRNA Accumulation,” J Exp Med 173:721 (1991), which are hereby incorporated by reference). Induction of signal 1 without signal 2 may lead to a state of immune tolerance (Chen, L., et al., “Costimulation of Antitumor Immunity by the B7 Counterreceptor for the T Lymphocyte Molecules CD28 and CTLA-4,” Cell 71:1093 (1992); Guinan, E. C., et al., “Pivotal Role of the B7:CD28 Pathway in Transplantation Tolerance and Tumor Immunity,” Blood 84:3261 (1994); Linsley, P. S., et al., “Binding of the B Cell Activation Antigen B7 to CD28 Costimulates T Cell Proliferation and Interleukin 2 mRNA Accumulation,” J Exp Med 173:721 (1991); Vassiliki, B., et al., “B7 but not Intercellular Adhesion Molecule-1 Costimulation Prevents the Induction of Human Alloantigen-Specific Tolerance,” J. Exp. Med. 178:1753 (1993), which are hereby incorporated by reference). B7 represents a family of at least several Ig-like molecules. These molecules are highly conserved among species, and both mouse and human B7.1 can stimulate CD28 counterreceptors of either species (Guinan, E. C., et al., “Pivotal Role of the B7:CD28 Pathway in Transplantation Tolerance and Tumor Immunity,” Blood 84:3261 (1994), which is hereby incorporated by reference). B7.1 Transfection into Nonimmunogenic Tumor Cells May Elicit a T-cell-Mediated Immune Response targeted Against Transfected (B7+) as well as Nontransfected (B7−) Tumor Cells (Baskar, S., et al., “Constitutive Expression of B7 Restores Immunogenicity of Tumor Cells Expressing Truncated Major Histocompatibility Complex Class II Molecules,” Proc Natl Acad Sci USA 90:5687 (1993); Chen, L., et al., “Tumor Immunogenicity Determine the Effect of B7 Costimulation on T cell-Mediated Tumor Immunity,” J Exp Med 179:523 (1994); Dohring, C., et al., “T-helper Accessory-Cell-Independent Cytotoxic Responses to Human Tumor Cells Transfected with a B7 Retroviral Vector,” Int J Cancer 57:754 (1994); Townsend, S. E., et al., “Specificity and Longevity of Antitumor Immune Responses Induced by B7-Transfected Tumors,” Cancer Res 54:6477 (1994), which are hereby incorporated by reference). A B7-mediated response has been specifically observed against ovarian carcinoma cells and melanoma cells (Dohring, C., et al., “T-helper Accessory-Cell-Independent Cytotoxic Responses to Human Tumor Cells Transfected with a B7 Retroviral Vector,” Int J Cancer 57:754 (1994), which is hereby incorporated by reference).

Expression of the B7.1 costimulatory ligand for CD28 on tumor cells via gene transfer has been shown to confer immunity to transduced as well as parental tumor cells (Baskar, J., et al., “Constitutive Expression of B7 Restores Immunogenicity of Tumor Cells Expressing Truncated Major Histocompatibility Complex Class II Molecules,” Proc Natl Acad Sci USA 90:5687 (1993); Chen, L., et al., “Tumor Immunogenicity Determine the Effect of B7 Costimulation on T cell-Mediated Tumor Immunity,” J Exp Med 179:523 (1994); Dohring, C., et al., “T-helper Accessory-Cell-Independent Cytotoxic Responses to Human Tumor Cells Transfected with a B7 Retroviral Vector,” Int J Cancer 57:754 (1994), which are hereby incorporated by reference).

The T-cell arm of the immune response can be focused by using antibodies to selectively “label” tumor cells and amplify the host response to other tumor associated neoantigens. Fusions with antibodies are described which recognize known antigens (e.g., CEA, her2/neu), but whose amino or carboxy terminal domains have been linked to or replaced by the B7.1 “costimulatory ligand” for the CD28 receptor involved in T-cell activation and/or the chemokine RANTES, to facilitate T-cell recruitment (See FIG. 1).

The costimulatory ligand is preferentially fused to the amino terminus of either the heavy or light chain of an antibody molecule. A more preferred embodiment is where the costimulatory ligand is fused to the amino acid terminus of the heavy chain.

Another embodiment of the invention is a method for stimulating a tumor specific immune response. The chimeric molecule having a binding domain capable of binding to a tumor cell associated antigen and a costimulatory ligand or active fragment of a costimulatory ligand may also be administered to a mammal. The chimeric molecule may also be produced in the mammal's cells. A gene encoding the chimeric molecule is introduced into the mammal and the chimeric molecule is then expressed from the gene.

The chimeric molecule that has a costimulatory ligand can be combined with a pharmaceutically-acceptable carrier in a composition to be administered to a mammal.

Another aspect of the invention is a method for stimulating a tumor specific immune response by administering both a chimeric molecule comprising a binding domain capable of binding to a tumor cell associated antigen and a chemokine or active fragment of a chemokine and a chimeric molecule comprising a binding domain capable of binding to a tumor cell associated antigen and a costimulatory ligand or active fragment of a costimulatory ligand to a mammal.

A composition for stimulating a tumor specific immune response is also provided that has chimeric molecules that have a chemokine connected to a tumor specific antibody, a costimulatory ligand connected to a tumor specific antibody, and a pharmaceutically-acceptable carrier.

The chemokine RANTES may also aid in activation of the recruited effector cell. Chimeric molecules are disclosed herein with binding domains directed against her2/neu, linked to sequences encoding the chemokine RANTES or to the extracellular domain of the B7.1 T cell costimulatory ligand. Combination with RANTES is designed to increase transendothelial migration and recruitment of immune effector cells, while combination with B7.1 is expected to amplify a specific host response to tumors from the RANTES mobilized effector cell population. Using this approach a costimulatory and/or chemokine stimulus can be delivered to the tumor site in vivo, and thereby elicit a beneficial anti-tumor specific immune response.

Globally, the strategy could also be applied as an alternative therapy against any potentially immunogenic tumor cell. Chimeric proteins are effective means of activating an immune response, making the strategy a powerful technique as an alternative therapeutic approach for a wide variety of tumors and other diseases.

In addition to cytokines and costimulatory molecules, other cytokines and adhesion molecules may also be utilized in the present invention. Chemokines are proinflammatory cytokines that are chemoattractants and activators of specific types of leukocytes and have been identified as playing a significant role in many disease states. Cellular adhesion molecules (such as selecting, integrins and their ligands) are involved in the intracellular interactions.

In addition to fusing a chemokine, cytokine, or costimulatory compound to the binding domain, the invention provides chimeric molecules having more than one T-cell effector molecule. T-cell effector molecules are molecules or fragments of molecules which effect the ability of T-cells to attack target cells. T-cell effector cells may function by recruiting T-cells to the area of the target cell or by activating T-cells. These chimeric molecules suitable for stimulating a tumor specific immune response have a binding domain capable of specifically binding to a tumor cell associated antigen and two or more T-cell effectors. T-cell effectors include chemokines, cytokines, and costimulatory molecules. Active fragments of T-cell effector molecules may also be used. The T-cell effectors are associated with the binding domain such that the binding domain remains capable of binding the tumor cell associated antigen and the T-cell effectors retain activity. Preferred combinations of T-cell effectors are: a chemokine and a costimulatory molecule, a chemokine and a cytokine, and a cytokine and a costimulatory molecule. Another preferred embodiment is where the chimeric molecule has three T-cell effectors; a chemokine, a cytokine and a costimulatory molecule. The preferred chemokine, cytokine and costimulatory molecules are RANTES, IL-2, and B7.1, respectively.

Another embodiment of the invention is a method for stimulating a tumor specific immune response by administering to a mammal a chimeric molecule having a binding domain capable of binding to a tumor cell associated antigen and two or more of the following: a chemokine, a cytokine, a costimulatory ligand, or an active fragment of any of the preceding compounds.

### EXAMPLES

### Example 1

Materials and Methods

Cell lines and reagents: SKBR3, THP-1, EL4, Sp2/0 and P3X63-Ag.653 cells were obtained from the American Type Culture Collection. Sp2/0, P3X63Ag8.563 and EL4 cells were cultured in Iscove's medium supplemented with 5% fetal bovine serum, L-glutamine, penicillin and streptomycin (GPS). SKBR3 and THP-1 cells were maintained in RPMI medium containing 10% fetal bovine serum and GPS. Recombinant human RANTES (rRANTES) was obtained from R&D Systems (Minneapolis, Minn.).

Antibody Expression vectors: For the construction of a humanized Her2.IgG3 antibody, the variable light and heavy chain sequences were obtained from the humanized humAb4D5-8 antibody (kindly provided by Dr. P. Carter, Genentech Inc., San Francisco, Calif.) (Carter, P., et al., “Humanization of an Anti-p185HER2 Antibody for Human Cancer Therapy”, Proc Natl Acad Sci USA, 89:4285-9 (1992); Rodrigues, M. L., et al., “Engineering a Humanized Bispecific F(ab′)2 Fragment for Improved Binding to T Cells,” Int J Cancer Suppl, 7:45-50 (1992), which are hereby incorporated by reference) and cloned into previously described mammalian expression vectors for human kappa light chain and IgG3 heavy chains, respectively (Shin, S. U., et al., “Expression and Characterization of an antibody binding specificity Joined to Insulin-like Growth Factor-1: Potential Applications for Cellular Targeting”, Proc Natl Acad Sci USA, 87:5322-6 (1990), which is hereby incorporated by reference). To construct RANTES.Her2.IgG3, human RANTES sequences were amplified from the plasmid pBS-RANTES (a generous gift from T. Schall ChemoCentryx, Mountain View, Calif.) using the sense primer 5′-GGCATAAGCTTGATATCTGAAGCCATGGGC-3′ (SEQ ID No. 1) and antisense primer 5′-GCGCGGTTAACCGTTATCAGGAAAATGC-3′ (SEQ ID No. 2), and the PCR product was subcloned as a HindIII/Hpal fragment at the 5′ end of a cassette encoding the (Ser-Gly₄)₃ linker sequences fused to the anti-HER2/neu V_{H} sequences. The resulting RANTES-linker-V_{H} coding sequences were isolated as an EcoRV/NheI fragment and cloned into an expression vector for human IgG3 heavy chain (Coloma, M. J., et al., “Novel vectors for the Expression of Antibody Molecules Using Variable Regions Generated by Polymerase Chain Reaction,” J Immunol Methods, 152:89-104 (1992), which is hereby incorporated by reference).

Recombinant antibody expression, immunoprecipitation and purification: Transfection, expression and purification of the recombinant antibodies were performed as previously described to obtain both Her2.IgG3 referred to as IgG3, and the RANTES anti-HER2/neu fusion protein referred to as RANTES.Her2.IgG3 (Shin, S. U., et al., “Expression and Characterization of an Antibody Binding Specificity Joined to Insulin-like Growth Factor-1: Potential Applications for Cellular Targeting”, Proc Natl Acad Sci USA, 87:5322-6 (1990), which is hereby incorporated by reference). Briefly, Sp2/0 or P3X63-Ag.653 myeloma cells were transfected with 10 μg of each of the anti-HER2/neu light chain and heavy chain expression vectors by electroporation. Transfected cells were plated at 10⁴ cells/well in 96-well U-bottom tissue culture plates and selected in 0.5 mM histidinol (Sigma Chemical Co., St. Louis, Mo.). Wells were screened for antibody secretion using a human IgG specific ELISA and positive wells expanded.

To determine the size of the secreted recombinant IgG3 and RANTES.Her2.IgG3 antibodies, supernatants from Sp2/0 cells grown overnight in medium containing ³⁵S-methionine (Amersham Corporation, Arlington Heights, Ill.) were immunoprecipitated with goat anti-human IgG (Zymed Laboratories Inc., San Francisco, Calif.) and staphylococcal protein A (IgGSorb, The Enzyme Center, Malden, Mass.). Precipitated antibodies were analyzed on SDS-PAGE gels in the presence or absence of the reducing agent β-mercaptoethanol. For purification of IgG3 and RANTES.Her2.IgG3, high producing clones were expanded in roller bottles in Hybridoma Serum-Free Medium (GIBCO), and 2-4 liters of cell-free media collected. Culture supernatants were passed through a GammaBind protein G column (Pharmacia Biotech Inc., Piscataway, N.J.) and the column washed with 10 mls of PBS. The proteins were successively eluted with a total of 10 mls of 0.1M glycine at pH 2.5 and pH 2.0, and the eluate neutralized immediately with 2M Tris-HCL pH 8.0. Eluted fractions were dialyzed against PBS and concentrated using Centricon filters with a molecular weight cut-off 50,000 Da (Amicon Inc., Beverly, Mass.).

Flow cytometry studies: SKBR3 cells were detached by treatment with 0.5 mM EDTA, washed and incubated with 10 μg/ml IgG3 or RANTES.Her2.IgG3 antibodies for 1-2 hours at 4° C., washed and stained with FITC-conjugated anti-human IgG (Sigma), or alternatively with biotin-conjugated anti-human RANTES (R&D Systems) followed by streptavidin-phytoerythrin (Sigma) and analyzed by flow cytometry.

Affinity analysis: The affinity of RANTES.Her2.IgG3 for its HER2/neu antigen was compared to that of the parental IgG3 antibody using an IAsys Optical Biosensor (Fisons Applied Sensor Technology, Paramus, N.J.). Soluble HER2/neu antigen (ECD, generously provided by Genentech Inc.) was immobilized on a sensitized microcuvette according to the manufacturer's instructions. Antibodies at 1×10⁻⁷ M concentration diluted in PBS with 0.05%. Tween-20 were added to the cuvette and association and dissociation rates measured. Rate constants were calculated using the FASTfit software (Supplied with IASYS System) as previously described (Coloma, M. J., et al., “Design and Production of Novel Tetravalent Bispecific Antibodies [see comments],” Nat Biotechnol, 15:159-63 (1997), which is hereby incorporated by reference).

F-actin polymerization studies: THP-1 cells, at 1×10⁶ cells/ml, were stimulated with cAMP at 1 μM for 72 hrs. Stimulated cells were washed and incubated with either recombinant RANTES (rRANTES), RANTES.Her2.IgG3 or control IgG3. Reactions were stopped at time points 0, 0.5, 1, 3, 5 and 10 minutes by fixing the cells in paraformaldehyde for more than 48 hours as previously described (Sham, R. L., et al., “Signal Transduction and the Regulation of Actin Conformation During Myeloid Maturation: Studies in HL60 Cells,” Blood, 77:363-70 (1991), which is hereby incorporated by reference). Fixed cells were stained with NBD-phallacidin (Molecular Probes, Eugene, OR) and analyzed by flow cytometry. The relative increase in Fluorescence over control time 0 was plotted.

Transendothelial migration assays: Human umbilical vein endothelial cells (HUVECs) were obtained from term umbilical cords through the courtesy of Dr. Lee Ann Sporn (University of Rochester, Rochester, N.Y.). Umbilical cords were flushed with Lactated Ringer's solution injected with Pronase (Calbiochem, San Diego, Calif.) and incubated for 20 minutes, after which the endothelial cells were flushed from the vein. First passage HUVECs were cultured in McCoy's 5A medium (GIBCO-BRL) supplemented with 20% FSB, 50 μg/ml endothelial mitogen (Biomedical Technologies Inc., Stoughton, Mass.) and 100 μg/ml heparin (Sigma in flasks preccated with 1% porcine gelatin (Sigma). At confluence, cultures were detached with trypsin/EDTA (GIBCO-BRL) washed and plated in Iscove's medium supplemented with 15% FBS, 15% horse serum, 180 ng/ml hydrocortisone (Sigma), 100 μg/ml endothelial growth factor (Biosource International, Camarillo, Calif.), 50 μg/ml heparin, 1% L-glutamine and 1% Penicillin-streptomycin on a 3 μm porous membrane insert of a transwell plate (Costar, Cambridge, Mass.). All HUVECs used in these studies are early passage cells (p3-p5). Transendothelial migration experiments were performed when HUVECs reached confluence following plating (approximately 2-3 days) using methods adapted from Mohle et al. (Mohle, R., et al., “Transendothelial Migration of CD34+ and Mature Hematopoietic Cells: An In Vitro Study Using a Human Bone Marrow Endothelial Cell Line,” Blood, 89:72-80 (1997), which is hereby incorporated by reference). Primary T-cells were purified from Ficoll-Hypaque separated peripheral blood mononuclear cells of normal donors using T-Cell enrichment columns (R&D Systems). They were plated over the HUVEC monolayer in the upper well of a transwell plate in X-Vivo 10 serum-free medium (BioWhittaker Inc., Walkersville, Md.). rRANTES, RANTES.Her2.IgG3 or IgG3 control were diluted in X-Vivo 10 medium and added in the lower wells. The plates were incubated at 37° C. for 24 hours, and cells that migrated to the lower well were counted using a hemocytometer. In another set of experiments, SKBR3 cells were preincubated with 10 μg/ml of either IgG3 or RANTES.Her2.IgG3 for 2 hours at 4° C. The cells were then washed three times, resuspended in X-Vivo 10 medium and plated in the lower well of the transwell plate at 2-4×10⁴ cells per well and transendothelial migration assay performed as described above.

### Example 2

Antibody Fusion Protein Design and Expression

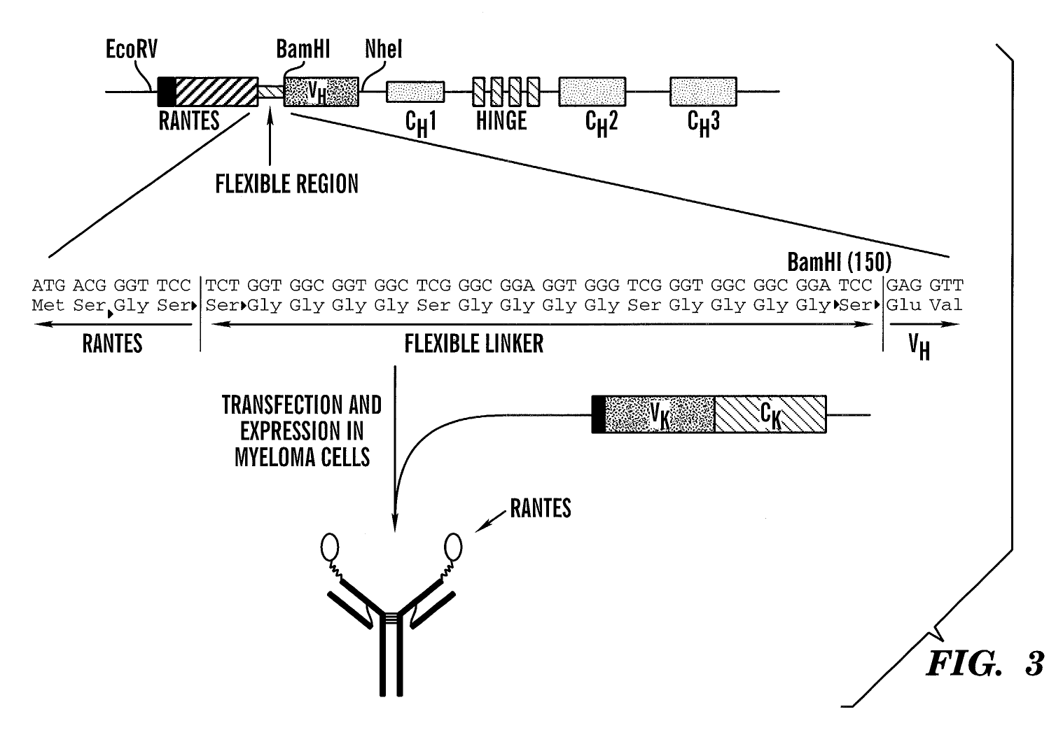

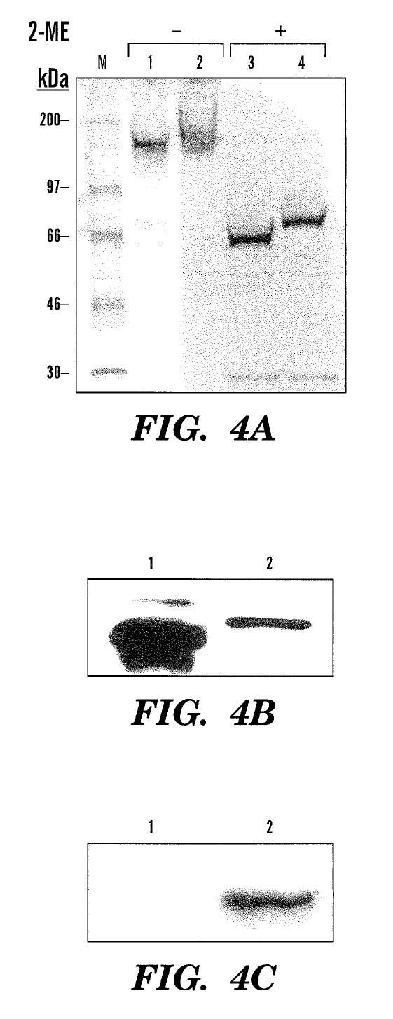

The antibody fusion protein RANTES.Her2.IgG3 was designed and constructed so that the chemokine RANTES was linked to the amino terminus of the heavy chain of the humanized anti-HER2/neu heavy chain antibody via a (Ser-Gly₄)₃ flexible linker (See FIG. 3). Expression vectors encoding the anti-HER2/neu light chain and the RANTES.Her2.IgG3 heavy chain were transfected into Sp2/0 myeloma cells, and stable transfectants identified and expanded. Recombinant protein was purified using a protein G affinity column. Assembly and secretion of the H₂L₂ form of the recombinant fusion protein was verified by SDS-polyacrylamide gel electrophoresis. A complete H₂L₂form (˜185 kDa) of the RANTES.Her2.IgG3 fusion protein is secreted by the myeloma cells (FIG. 4A, lane 2). Following reduction of 2-mercaptoethanol, both RANTES.IgG# heavy chain (FIG. 4A, lane 4), which has higher apparent MW than the IgG3 heavy chain (FIG. 4A, lane 3) and intact anti-HER2/neu light chain (˜25 kDa) were detected. Both Her2.IgG3 and RANTES.Her2.IgG3 recombinant antibodies were detected with an anti-human IgG antibody (FIG. 4B), whereas only RANTES.Her2.IgG3 was specifically detected with an anti-RANTES antibody (FIG. 4C).

### Example 3

Characterization of the Binding Domains of RANTES.Her2.IgG3 Fusion Protein

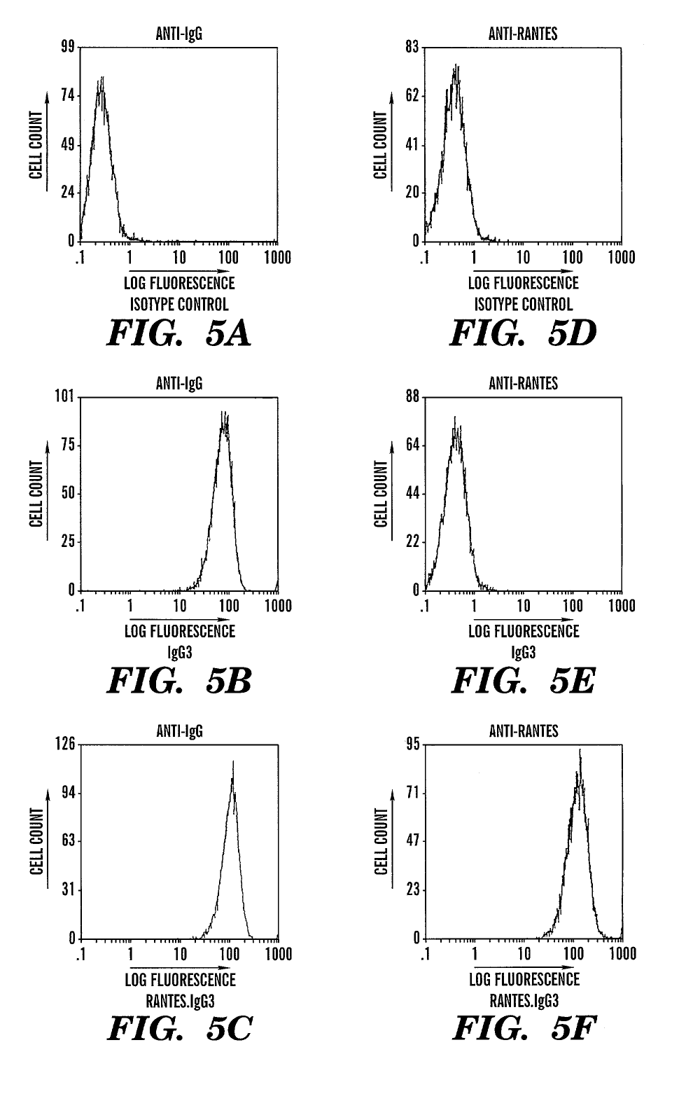

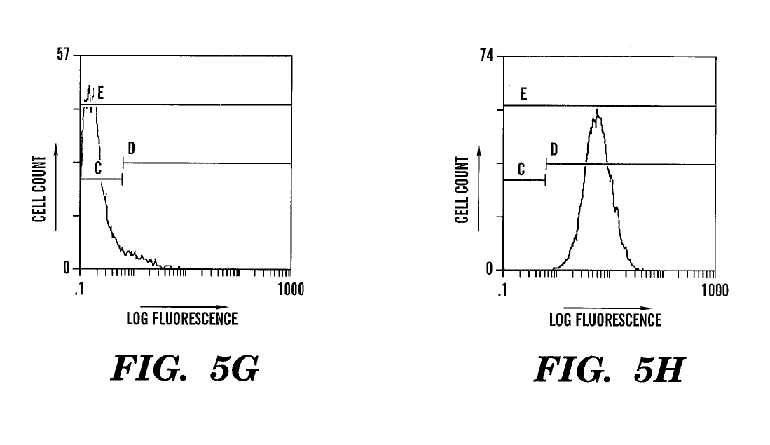

To test the ability of recombinant RANTES.Her2.IgG3 to bind to the HER2/neu antigen, SKBR3 cells a breast cancer cell line known to express high levels of HER2/neu, were incubated with either an isotype control human IgG3 (anti-dansyl IgG3), Her2.IgG3 or RANTES.Her2.IgG3. Cells were then stained with either FITC-conjugated anti-human IgG, or with biotin-conjugated anti-RANTES antibody followed by PE-conjugated streptavidin, and analyzed by flow cytometry. Both Her2.IgG3 (FIGS. 5b & e) and RANTES.Her2.IgG3 (FIGS. 5c & f) bound specifically to SKBR3 cells. Therefore, fusion of the extracellular domain of RANTES to the amino terminus of Her2.IgG3 did not interfere with recognition of the HER2/neu antigen by the antibody domain. SKBR3 cells incubated with RANTES.Her2.IgG3 also stained positively with anti-human RANTES indicating that after binding of RANTES.Her2.IgG3 to antigen, the RANTES domain was still accessible to antibody (FIG. 5f). The same experiment was repeated using EL4 cells stably expressing the human HER2/neu antigen by gene transfer. Binding to cell surface HER2/neu antigen was detected by flow cytometry on EL4Her2 cells (FIG. 5h), while no binding was detected on parental cells which did not express the HER2/neu antigen (FIG. 5g).

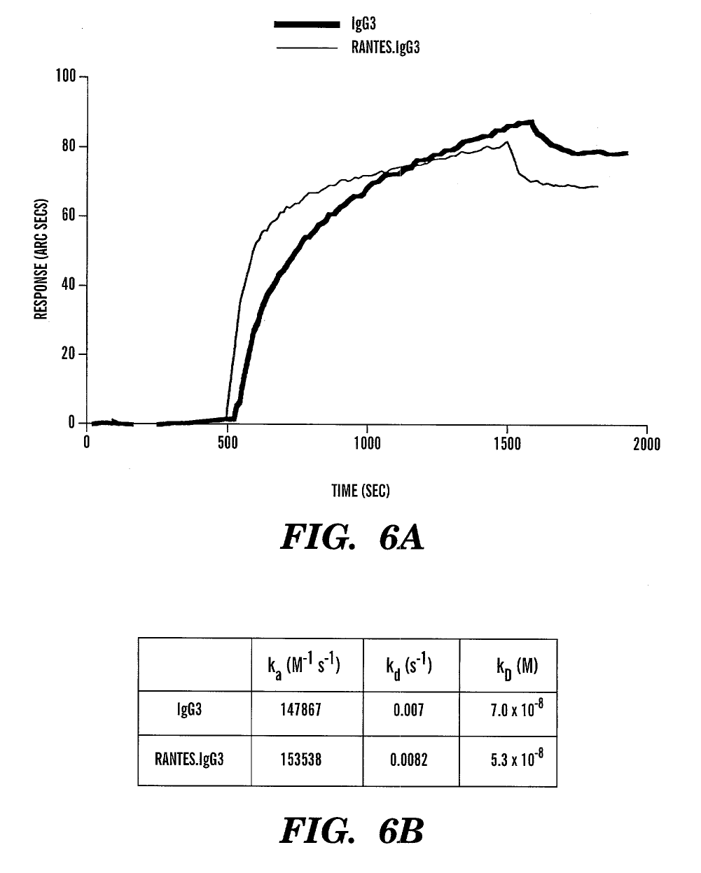

The affinities of RANTES.Her2.IgG3 and Her2.IgG3 for antigen were directly compared using an IAsys Biosensor (FIG. 6). The soluble extracellular domain of HER2/neu (ECD) was immobilized on a microcuvette as described in Materials and Methods. Her2.IgG3 or RANTES.Her2.IgG3 was added to the ECD coated cuvette, and the association and dissociation rate constants determined. The affinity (K_{D}) of RANTES.Her2.IgG3 was 5.3×10⁻⁸ M, similar to the affinity 7.0×10⁻⁸ M determined for the parental Her2.IgG3. These studies indicate that fusion of the low molecular weight RANTES molecule at the amino terminus of the Her2.IgG3 heavy chain did not appreciably alter the affinity of the anti-HER2/neu antibody for its antigen.

### Example 4

RANTES.Her2.IgG3 Transmits a Chemotactic Signal

The chemotactic effect of RANTES is accompanied by a change in the configuration of intracellular actin in the cytoskeleton. An F-actin polymerization assay was used to study the biological effect of RANTES.Her2.IgG3 fusion protein (Sham, R. L., et al., “Signal Pathway Regulation of Interleukin-8-induced Actin Polymerization in Neutrophils,” Blood, 82:2546-51 (1993), which is hereby incorporated by reference). In this assay, c-AMP differentiated THP-1 monocytic cells were treated with either parental Her2.IgG3 antibody, RANTES.Her2.IgG3 fusion protein or rRANTES (FIG. 7). Aliquots of the treated cells were harvested at 0.5, 1, 3, 5 and 10 minutes, fixed, and stained with NBD-phallacidin which detects polymerized actin. RANTES.Her2.IgG3 induced F-actin polymerization within 0.5 minutes of treatment and the polymerization response was maintained for about 3 minutes, while Her2.IgG3 alone did not increase F-actin content. The polymerization curve obtained with RANTES.Her2.IgG3 was similar to that observed with rRANTES. The F-actin response obtained with RANTES.Her2.IgG3 is therefore mediated by the RANTES domain of the fusion protein and not the IgG3 domain.

### Example 5

RANTES.Her2.IgG3 Mediates Transendothelial Migration of T Cells

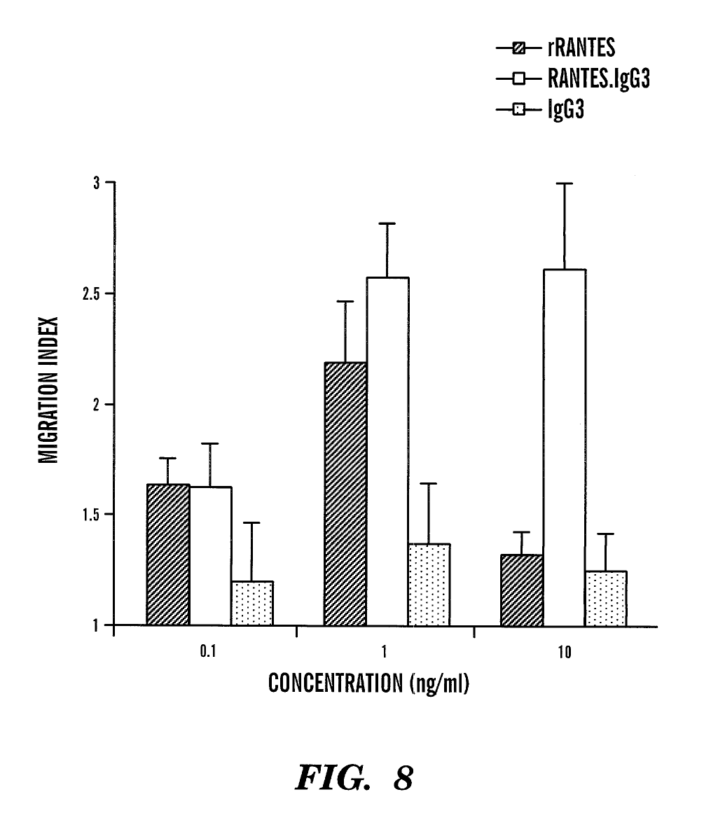

To determine whether RANTES.Her2.IgG3 fusion protein could facilitate transendothelial migration of effector cells, a modified Boyden-Chamber chemotaxis assay was used. HUVEC monolayers were grown to confluence on the culture insert of a transwell culture plate. Migration of primary peripheral blood T cells plated in the upper well was studied in response to different concentrations of RANTES.Her2.IgG3 or rRANTES added to the lower well. Table 1 summarizes the data from four different experiments, and the average migration index of all experiments is plotted in FIG. 8. The chemotactic response of purified peripheral blood T cells to RANTES.Her2.IgG3 was similar to that observed with rRANTES. Significant migration of T cells was observed in response to RANTES.Her2.IgG3 at 1.0 and 10.0 ng/ml compared to control IgG3 (p=0.0133 and 0.0062 respectively). Therefore, the chemotactic response is mediated by the RANTES domain of the RANTES.Her2.IgG3 fusion protein.

Table 1 Transendothelial migration of peripheral blood T cells in response to RANTES.Her2.IgG3. A HUVEC monolayer was grown to confluence on the porous membrane of a transwell plate (see Materials and Methods). Peripheral blood T cells were purified from blood obtained from normal donors and plated in the upper well of the transwell plate. rRANTES, RANTES.Her2.IgG3 or IgG3 were added to the lower wells at the indicated concentrations. Migration was allowed to proceed at 37° C. for 24 hours, and the number of migrated cells in the lower well was counted and recorded as % migration.

TABLE 1
Transendothelial migration of peripheral blood T cells in response to
RANTES.Her2.IgG3. A HUVEC monolayer was grown to confluence on the porous membrane of a
transwell plate (see Materials and Methods). Peripheral blood T cells were purified from
blood obtained from normal donors and plated in the upper well of the transwell plate.
rRANTES, RANTES.Her2.IgG3 or IgG3 were added to the lower wells at the indicated
concentrations. Migration was allowed to proceed at 37° C. for 24 hours, and the number of
migrated cells in the lower well was counted and recorded as % migration.

| | | % Migration (migration index)* | | | | |
|---|---|---|---|---|---|---|
| | | Experiment | Experiment | Experiment | Experiment | Average Migration |
| Condition | ng/ml | #1 | #2 | #3 | #4 | Index ± SEM |
| None | — | 129 (1.0) | 16.4 (1.0) | 5.2 (1.0) | 5.9 (1.0) | 1.0 ± 0.00 |
| rRANTES | 0.1 | 24.9 (1.9) | 23.0 (1.4) | 9.2 (1.8) | 8.4 (1.4) | 1.6 ± 0.13 |
| rRANTES | 1.0 | 34.4 (2.7) | 27.5 (1.7) | 14.3 (2.8) | 9.9 (1.7) | 2.2 ± 0.30 |
| rRANTE5 | 10.0 | ND | ND | 7.6 (91.6) | 6.8 (1.2) | 1.3 ± 0.11 |
| RANTES.Her2.IgG3 | 0.1 | 28.2 (2.2) | 26.7 (1.6) | 6.7 (1.3) | 8.1 (1.4) | 1.6 ± 0.20 p = 0.2665^{‡} |
| RANTES.Her2.IgG3 | 1.0 | 38.3 (3.0) | 34.4 (2.1) | 15.5 (3.0 | 13.3 (2.2) | 2.6 ± 0.24 p = 0.0133^{‡} |
| RANTES.Her2.IgG3 | 10.0 | 44.9 (3.5) | 6.2 (1.6) | 15.1 (2.9) | 14.3 (2.4) | 2.6 ± 0.40 p = 0.0062^{‡} |
| IgG3 | 0.1 | 18.5 (1.4) | 6.7 (0.4) | 6.6 (1.3) | 9.6 (1.6) | 1.2 ± 0.27 |
| IgG3 | 1.0 | 18.1 (1.4) | 10.0 (0.6) | 9.6 (1.9) | 9.6 (1.6) | 1.4 ± 0.27 |
| IgG3 | 10.0 | 18.1 (1.4) | 6.7 (0.4) | 8.9 (1.7) | 5.9 (1.0) | 1.1 ± 0.28 |

*= Values correspond to percent of T cells placed in the upper well (1 × 10⁵ in experiment #1, 4.7 × 10⁴ in experiment #2, 2 × 10⁵ in experiments #3 and 4) which migrate to the lower well in response to the described conditions. In parenthesis, the migration index is shown for each experiment calculated as % migration for a specific condition divided by control % migration in medium only.
^{‡}= the p value was calculated from paired t-test comparing RANTES.IgG3 to IgG3 in all four experiments at the same concentration.

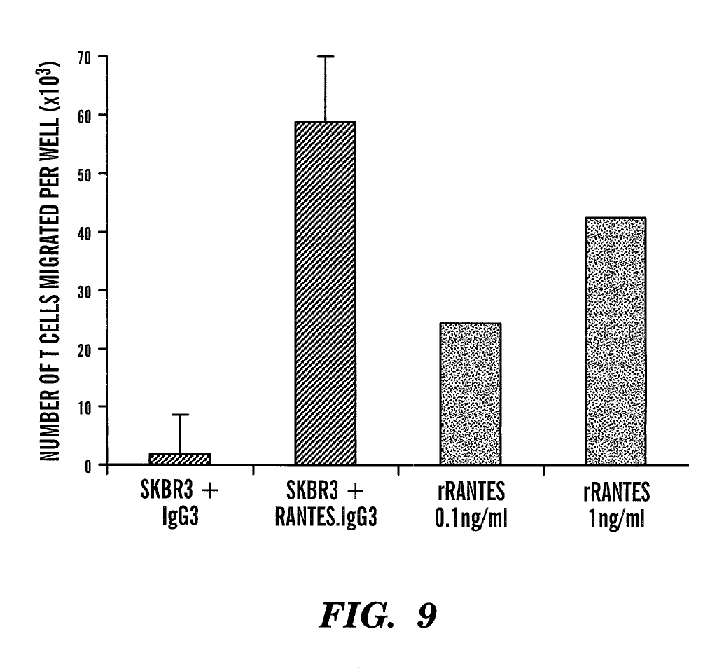

In another set of experiments, the ability of RANTES.Her2.IgG3 to induce migration was tested following binding to antigen on the surface tumor cells through the antibody domain. SKBR3 cells, which express high levels of HER2/neu, were preincubated with Her2.IgG3 or RANTES.Her2.IgG3, unbound protein was removed by washing, and the cells placed in the lower well of a chemotaxis transwell plate. Migration of peripheral blood T cells through a confluent HUVEC layer was measured 24 hours later as described above. RANTES.Her2.IgG3 but not IgG3 prebound to the cells was capable of inducing migration of T cells (See FIG. 9). Levels of migration with RANTES.Her2.IgG3 actually exceeded those seen in response to soluble rRANTES at 0.1 and 1 ng/ml. These experiments demonstrate that RANTES.Her2.IgG3 fusion protein bound to tumor cell surface was capable of creating a gradient necessary for its chemotactic activity in the vicinity of targeted tumor cells.

### Example 6

Materials and Methods

Cell lines and reagents: CHO, EL4, SKBR3, Sp2/0 and P3X63-Ag.653 cells were available in the laboratory or obtained from the American Type Cell Collection. E14, Sp2/0 and P3X63Ag8.563 cells were cultured in Iscove's medium supplemented with 5% fetal bovine serum, L-glutamine, penicillin and streptomycin (GPS). SKBR3 cells were grown in RPMI medium containing 10% fetal bovine serum and GPS. CHO cells were maintained in DMEM supplemented with 10% fetal bovine serum and GPS. CHO/CD28, CHO/B7 cells as well as the CD28Ig and B7Ig soluble proteins were kindly provided by Dr. P. Linsley (Bristol-Myers-Squibb Pharmaceutical Research Institute, Seattle, Wash.): CHO/CD28 and CHO/B7 cells were grown in the same media as CHO cells supplied with 0.2 mM proline and 1 μM methotrexate. CHO cells transfected with the HER2/neu CDNA were maintained under selection with 0.5 ng/ml of Geneticin (GIBCO/BRL, Gaithersburg, Md.). Soluble CTLA4Ig was purified from a hybridoma obtained from Dr. J. Allison (University of California at Berkeley, Calif.) using standard protein A column purification methods.

Expression vectors, HER2/neu retroviral vector and gene delivery: The plasmid encoding the human HER2/neu cDNA (clone 0483 generously provided by Genentech Inc., San Francisco, Calif.) was digested with HindIII, filled-in using Klenow polymerase, digested with XhoI and cloned into the XhoI and filled-in BamHI sites of the retroviral vector LXSN (Miller, D. et al., “Improved Retroviral Vectors for Gene Transfer and Expression,” Biotechniques 7:980-985 (1989), which is hereby incorporated by reference). The resulting plasmid was transfected into the PA317 packaging cell line using Lipofectin Reagent (GIBCO) and cells selected in 0.5 ng/ml Geneticin. Culture supernatant from the vector-producing PA317 cells was harvested, filtered through 0.45 μm filters and used to transduce CHO cells to derive CHO/HER2 cells.

Anti-HER2/neu kappa light chain expression vector: The light chain variable domain of the humanized humAb4D5-8 antibody was amplified from the plasmid pAK19 917) (kindly provided by Dr. P. Carter, Genentech Inc.) and fused to the 3′-end of human kappa leader sequence by overlapping polymerase chain reaction (PCR). The primers used in the first cycle of amplification are: (a) 5′-GGGGATATCCACCATGG(A/G)ATG(C/G)AGCTG(T/G)GT(C/A)AT(G/C)CTCTT-3′ (SEQ ID No. 3) and (b) 5′-GACTGGGTCATCTGGATGTCGGAGTGGACACCTGTGGAG-3′ (SEQ ID No. 4) for the leader sequence using a plasmid encoding human kappa light chain sequences as template DNA, (c) 5′-CTCCACAGGTGTCCACTCCGACATCCAGATGACCCAGT-3′ (SEQ ID No. 5) and (d) 5′-GCTTGTCGACTTACGTTTGATCTCCACCTTGG-3′ (SEQ ID No. 6) for the V_{L} sequences using pAK19 as template DNA. The resultant PCR products were mixed and used as template for the amplification with primers (a) and (d). The final PCR product of 470 bp was digested with EcoRV and SalI and cloned into the human kappa light chain expression vector previously described (Coloma, M. et al., “Novel Vectors for the Expression of Antibody Molecules Using Variable Regions Generated by Polymerase Chain Reaction,” J Immunol Methods 152:89-104 (1992), which is hereby incorporated by reference).

Anti-HER2/neu heavy chain expression vector: The strategy used to clone the heavy chain variable domain (V_{H}) from pAK19 is similar to the V_{L} cloning strategy. The primers used for amplification are: (a) 5′-GGGGATATCCACCATGG(A/G)ATG(C/G)AGCTG(T/G)GT(C/A)AT(G/C)CTCTT-3′ (SEQ ID No. 3), (b) 5′-GACTCCACCAGCTGAACCTCGGAGTGGACACCTGTGGAG-3′ (SEQ ID No. 7), (c) 5′-CTCCACAGGTGTCCACTCCGAGGTTCAGCTGGTGGAGT-3′ (SEQ ID No. 8), and (d) 5′ -TTGGTGCTAGCCGAGGAGACGGTGACCAG-3′ (SEQ ID No. 9). The final 500 bp PCR product encoding the leader fused to the V_{H} sequences of anti-HER2/neu was cloned as an EcoRV/NheI fragment into the human IgG3 mammalian expression vector previously described (Coloma, M. et al., “Novel Vectors for the Expression of Antibody Molecules Using Variable Regions Generated by Polymerase Chain Reaction,” J Immunol Methods 152:89-104 (1992), which is hereby incorporated by reference).

Anti-HER2/neu B7.her2.IgrG3 fusion heavy chain expression vector: The extracellular domain of the human B7.1 including the leader sequences were amplified using the primers 5′-GGCATAAGCTTGATATCTGAAGCCATGGGC-3′ (SEQ ID No. 1) and 5′-GCGCGGTTAACCGTTATCAGGAAAATGC-3′ (SEQ ID No. 2), and cloned as a HindIII/HpaI fragment at the 5′ end of the (Ser-Gly₄)₃ linker sequences into a pUC19-flex plasmid. The V_{H} domain of the humanized humAb4D5-8 antibody was amplified by polymerase chain reaction from the plasmid pAK19 using primers 5′-GGCGGCGGATCCGAGGTTCAGCTGGTG-3′ (SEQ ID No. 10) and 5′-TTGGTGCTAGCCGAGGAGACGGTGACCAG-3′ (SEQ ID No. 9), digested with BamHI and HpaI and cloned at the 3′ end of the B7.1 and flexible linker sequences. The resulting insert encoding the B7.1-linker-V_{H} sequences was isolated as an EcoRV/NheI fragment and cloned into the expression vector for the IgG3 heavy chain (Coloma, M. et al., “Novel Vectors for the Expression of Antibody Molecules Using Variable Regions Generated by Polymerase Chain Reaction,” J Immunol Methods 152:89-104 (1992), which is hereby incorporated by reference).

Recombinant antibody expression, immunoprecipitation and purification: Purified recombinant anti-HER2/neu antibody alone is referred to in the manuscript as Her2.IgG3, and the anti-HER2/neu antibody fused to B7.1 as B7.Her2.IgG3. Transfection, expression and purification of the recombinant antibodies were performed as described previously (Shin, S. et al., “Expression and Characterization of an Antibody Binding Specificity Joined to Insulin-Like Growth Factor 1: Potential Applications for Cellular Targeting,” Proc Natl Acad Sci USA 87:5322-6 (1990), which is hereby incorporated by reference). Briefly, non-secreting Sp2/0 or P3X63-Ag.653 myeloma cells were transfected with 10 μg of each of the anti-Her2/neu light chain and heavy chain expression vectors by electroporation. Transfected cells were plated at 10,000 cells per well in 96-well U-bottom tissue culture plates. The next day, selection in 0.5 mM histidinol (Sigma, St. Louis, Mo.) was initiated and maintained for 10-14 days. Wells were screened for antibody secretion by human IgG specific ELISA as previously described and positive wells expanded. To determine the size of the secreted recombinant antibodies supernatants from cells grown overnight in medium containing ³⁵S-methionine, were immunoprecipitated with goat anti-human IgG (Zymed Laboratories Inc., San Francisco, Calif.) and staphylococcal protein A (IgGSorb, The Enzyme Center, Maiden, Mass.). Precipitated antibodies were analyzed on SDS-polyacrylamide gels in either presence or absence of reducing agents. For purification of her2.IgG3 and B7.her2.IgG3 antibodies, antibody secreting Sp2/0 clones were expanded in roller bottles in Hybridoma Serum-Free Medium (GIBCO), and 2-4 liters of cell-free media collected. Culture supernatants were passed through a GammaBind protein G column (Pharmacia biotech Inc., Piscataway, N.J.) and the column washed with 10 mls of PBS. The protein was successively eluted with a total of 10 mls of 0.1M glycine at pH 4.0, pH 2.5 and pH 2.0, and the eluate neutralized immediately with 2M Tris-HCL pH 8.0. The eluted fractions were dialyzed and concentrated using Centricon filters with molecular weight cut-off of 30,000 Da (Amicon Inc., Beverly, Mass.).

Flow cytometry studies: Cells were detached by treatment with 0.5 mM EDTA, washed and incubated with recombinant her2.IgG3 or B7.her2.IgG3 antibodies for 1-2 hours at 4° C., washed and stained with FITC-conjugated anti-human IgG (Sigma), or PE-conjugated anti-human B7.1 (Beckton-Dickinson, San Jose, Calif.) and analyzed by flow cytometry.

Affinity analysis: The affinity of B7.her2.IgG3 fusion protein for the antigen was compared to that of the parental her2.IgG3 antibody using the IAsys Optical Biosensor from Fisons Applied Sensor Technology (Paramus, N.J.). Soluble HER2/neu antigen (ECD, generously provided by Genentech Inc.) was immobilized on a sensitized micro-cuvette according to the manufacturer's instructions. Her2.IgG3 or B7.her2.IgG3, at different concentrations in PBS with 0.05% Tween-20, were added to the cuvette and association and disassociation measured. Rate constants were calculated using the FASTfit software (Supplied with the IASYS System) as previously described (Coloma, M. et al., “Design and Production of Novel Tetravalent Bispecific Antibodies [See Comments],” Nat Biotechnol 15:159-63 (1997), which is hereby incorporated by reference).

T-cell proliferation assays: Human peripheral blood mononuclear cells were isolated from normal donor blood using standard Ficoll-hypaque density centrifugation. Human T-cell enrichment columns (R&D systems, Minneapolis, Minn.) were used for T-cell purification according to the manufacturer's instructions. Purified T cells were plated in flat-bottom 96-well tissue culture plates at 1×10⁵ cells per well in RPMI supplemented with 5% fetal bovine serum. Irradiated (5,000 rads) CHO, CHO/Her2 or CHO/B7 cells were added at 2×10⁴ cells per well in presence of 0, 1, 5 or 10 μg/ml recombinant her2.IgG3 or B7.her2.IgG3 and 10 ng/ml PMA (Sigma). Plates were incubated at 37° C. for 3 days, and pulsed with 0.5 μCi per well of ³H-thymidine for 16-18 hours, harvested and ³H-thymidine incorporation measured.

### Example 7

Design and Expression of the Recombinant Antibodies

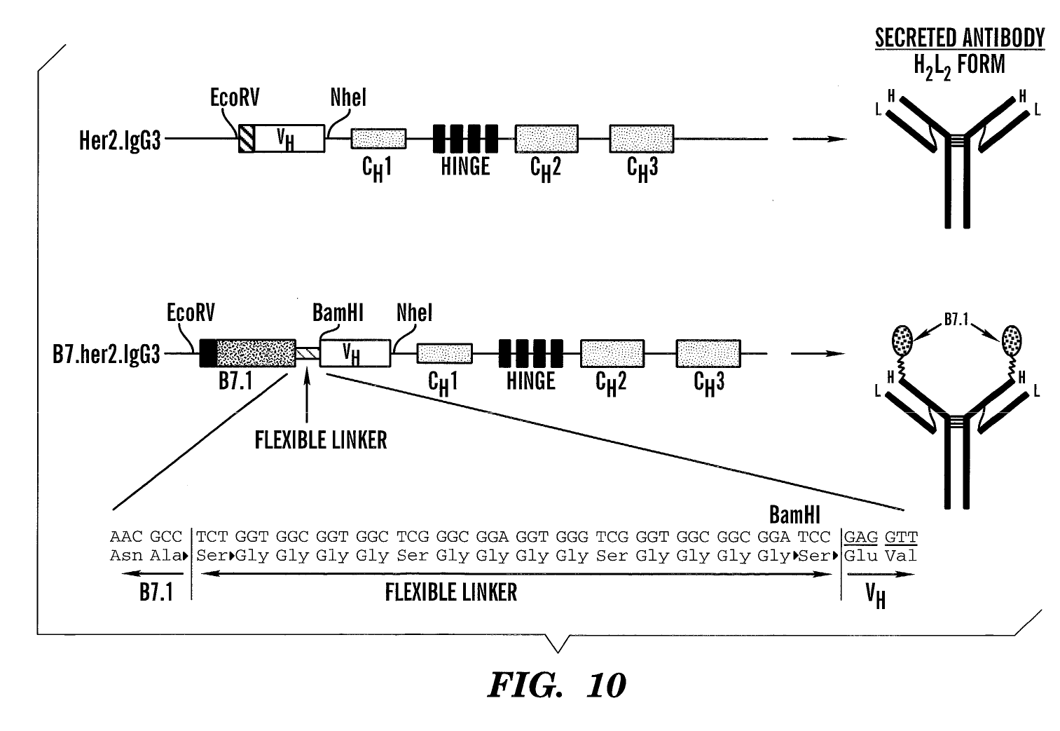

The expression vectors for the human IgG3 heavy and kappa light chains were previously described (Coloma, M. et al., “Novel Vectors for the Expression of Antibody Molecules Using Variable Regions Generated by Polymerase Chain Reaction,” J Immunol Methods 152:89-104 (1992), which is hereby incorporated by reference). The variable domains of the anti-HER2/neu antibody were amplified by PCR from the plasmid pAK19 (kindly provided by P. Carter, Genentech Inc.) (Carter, P. et al., “High Level Escherichia Coli Expression and Production of a Bivalent Humanized Antibody Fragment,” Biotechnology (10) 10:163-7 (1992), which is hereby incorporated by reference), and cloned into the corresponding heavy or light chain expression vectors to derive her2.IgG3. To construct a fusion antibody between her2.IgG3 and B7.1 (referred to as B7.her2.IgG3), the extracellular domain of human B7.1 was cloned at the 5′-end of the heavy chain variable region of her2.IgG3 (FIG. 10). A flexible (Ser-Gly₄)₃ linker was provided at the fusion site of the recombinant fusion protein to facilitate correct folding of both antibody and B7.1 domains. B7.1 was expressed at the amino terminus of the heavy chain because B7.1 fused to the carboxyl terminus of the C_{H}3 domain showed decreased affinity for CD28. These results are consistent with a critical role of the amino terminus of B7.1 in mediating its biological activity (Guo, Y. et al., “Mutational Analysis and an Alternatively Spliced Product of B7 Defines its CD28/CTLA4-Binding Site on Immunoglobulin C-Like Domain,” J Exp Med 181:1345-1355 (1995), which is hereby incorporated by reference). The light chain and either the her2.IgG3 or B7.her2.IgG3 heavy chain expression vectors were contransfected into Sp2/0 myeloma cells and stable transfectants secreting soluble proteins identified by ELISA.

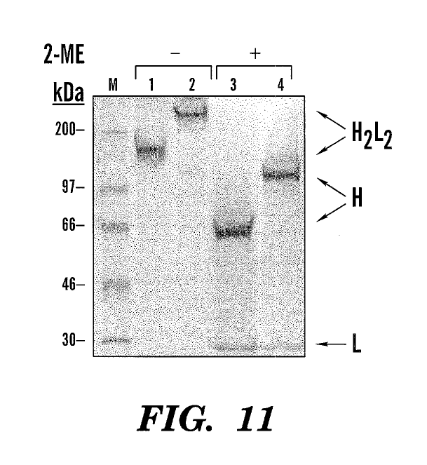

To determine the molecular weight and assembly of the transfected proteins, cells were grown overnight in ³⁵S-methionine and the secreted proteins immunoprecipitated and analyzed by SDS-PAGE. In the absence of reducing agents, her2.IgG3 migrates with an apparent molecular weight of 170 kDa while B7.her2.IgG3 is about 250 kDa (FIG. 11, lanes 1 and 2 respectively). Following treatment with 2-mercaptoethanol, light chains of 25kDa are seen for both proteins while her2.IgG3 has a heavy chain of approximately 60 kDa and B7.her2.IgG3 a heavy chain of approximately 100 kDa (FIG. 11, lanes 3 and 4 respectively). Therefore, proteins of the expected molecular weight are produced and the fusion of the extracellular domain of B7.1 to the her2.IgG3 heavy chain does not appear to alter secretion of the fully assembled H₂L₂ form of the antibody.

### Example 8

Antigen Binding

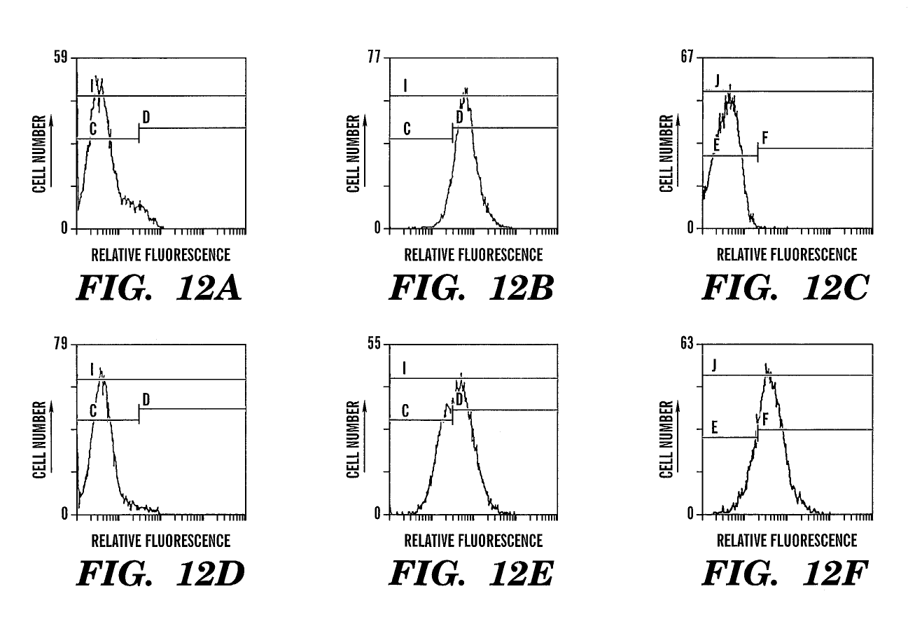

The ability of recombinant her2.IgG3 and B7.her2.IgG3 to bind to the HER2/neu antigenic target was tested by flow cytometry (See FIG. 12). CHO cells stably expressing the HER2/neu antigen (CHO/Her2) derived by retroviral-mediated gene transfer and non-transduced CHO cells were incubated with either her2.IgG3 or B7.her2.IgG3. Binding was assayed by staining with either FITC-conjugated anti-human IgG or PE-conjugated anti-human B7.1 antibodies followed by flow cytometry. Both her2.IgG3 (See FIGS. 12A & B) and B7.her2.IgG3 (See FIGS. 12D & E) bound specifically to CHO/Her2 and not to parental CHO cells. Therefore, fusion of the extracellular domain of B7.1 to a complete her2.IgG3 antibody resulted in a fusion antibody capable of specifically recognizing the HER2/neu antigen through the antibody domain. CHO/Her2 cells incubated with B7.her2.IgG3 also stained positively with anti-human B7.1 indicating that binding of B7.her2.IgG3 to the antigen through its antibody domain did not interfere with antibody recognition of the B7.1 fusion domain (FIG. 12F).

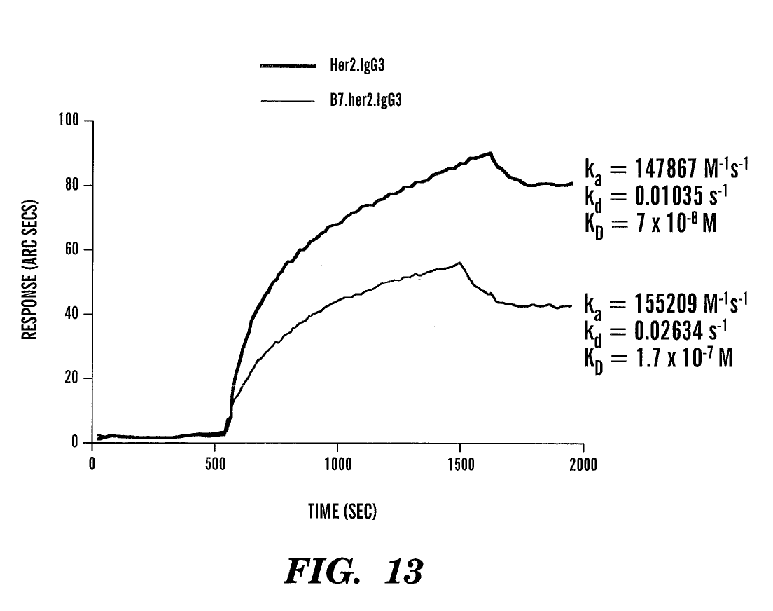

The affinities of the her2.IgG3 and B7.her2.IgG3 antibodies for the HER2/neu antigen were compared using the IAsys biosensor (FIG. 13). Her2.IgG3 or B7.her2.IgG3, at 1×10⁷ M concentration, were added to a cuvette with soluble HER2/neu antigen ECD immobilized on its surface and the association and dissociation measured as the samples were added and washed from the cuvette. The calculated affinity of 1.7×10⁻⁷ M for B7.her2.IgG3 was decreased about 2.5 fold compared to the affinity of 7×10⁻⁸ M obtained for the parental her2.IgG3. The modest decrease in affinity primarily reflected a reduction in the dissociation constant of B7.her2.IgG3.

### Example 9

B7.1 Binding Studies

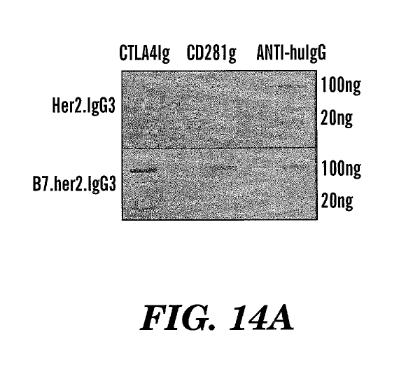

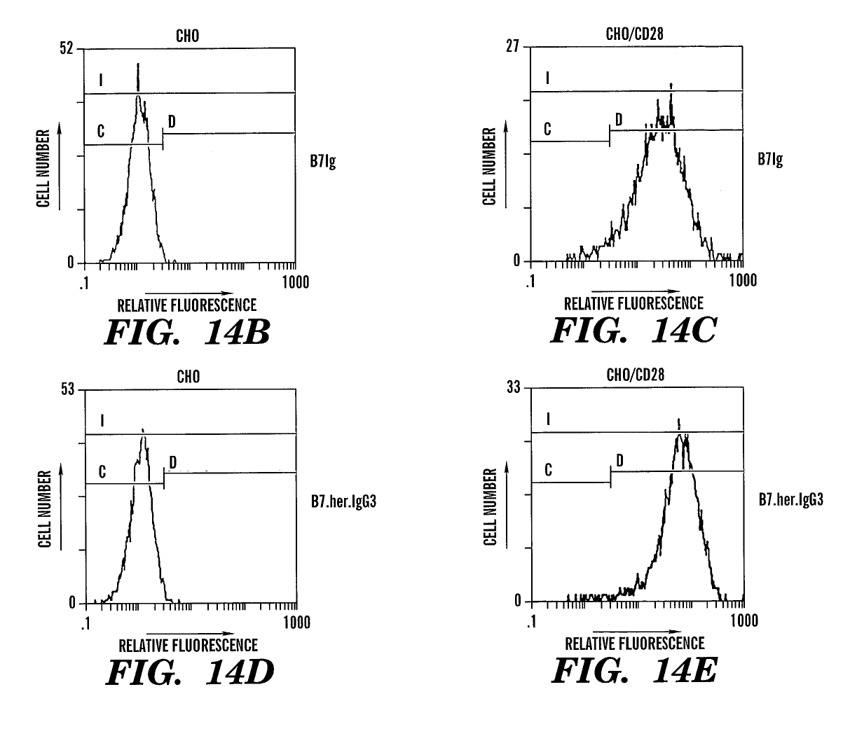

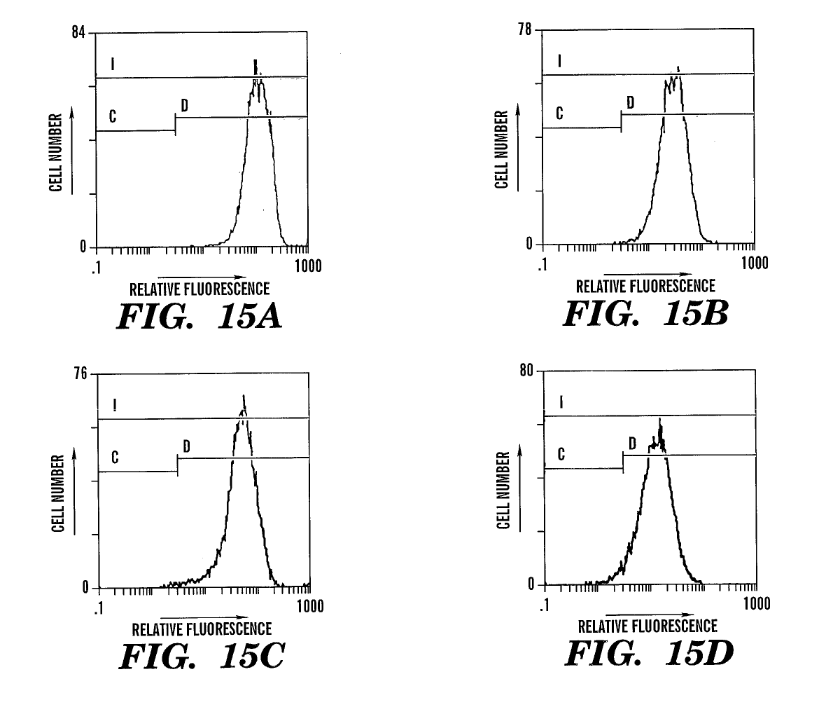

Ability of the B7.1 domain in the B7.her2.IgG3 fusion protein to bind to its receptors CTLA4 and CD28 was studied by two different methods. Soluble CTLA4Ig and CD28Ig immobilized on nitrocellulose membrane were incubated with either her2.IgG3 or B7.her2.IgG3 (FIG. 14A). Strong binding of B7.her2.IgG3 to CTLA4Ig was observed but no binding of her2.IgG3. B7.her2.IgG3 also bound CD28Ig although with a lesser affinity than to CTLA4Ig. This was expected since the reported affinity of B7.1 for CTLA4 is 20-fold higher than for CD28 (Linsley, P. et al., “CTLA-4 is a Second Receptor for the B Cell Activation Antigen B7,” J Exp Med 174:561-9 (1991), which is hereby incorporated by reference). In another experiment, CHO cells stably expressing CD28 were used to detect B7.her2.IgG3 binding (FIG. 14B). Parental CHO or CHO/CD28 cells were incubated with either B7Ig (a kind gift from Dr. P. Linsley) or B7.her2.IgG3, washed, and binding detected by staining with FITC-conjugated anti-human IgG followed by flow cytometry. Specific binding of B7.her2.IgG3 and B7Ig to CD28 present on CHO-CD28+ cells but not to control CHO cells was observed.

### Example 10

Stability of the Anti-HER2/neu Recombinant Antibodies on the Cell Surface

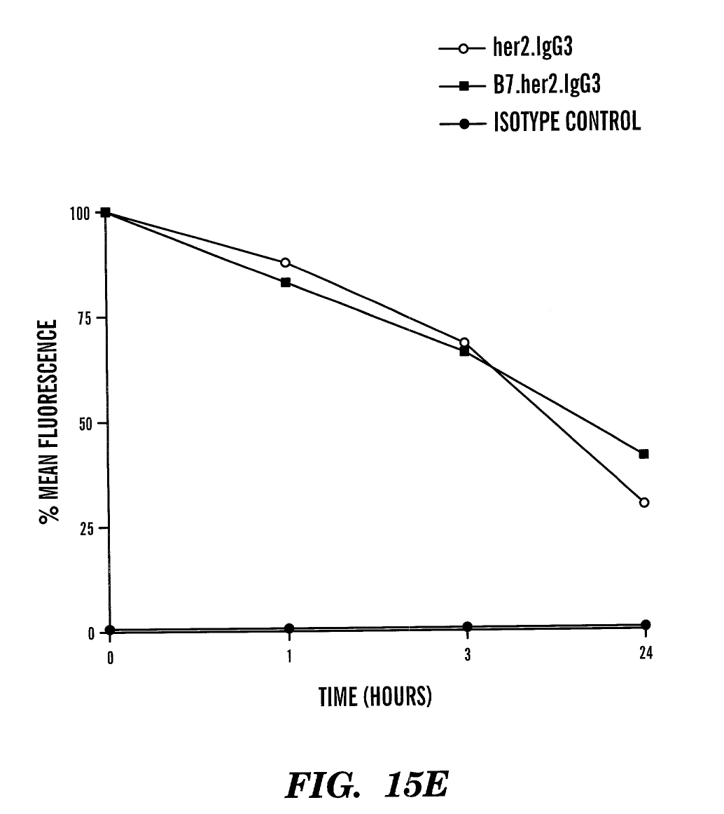

Since recruitment and activation of tumor specific T-cells would depend on the presence of B7.1 on the tumor cell surface, the stability of B7.her2.IgG3 bound to the HER2/neu antigen expressed on the cell membrane was characterized. SKBR3 cells, from a human breast cancer cell line known to express high levels of HER2/neu, were incubated with 10 μg/ml of either her2.IgG3 or B7.her2.IgG3 at 4° C. to allow maximum binding. The cells were then washed and incubated at 37° C. in culture medium. At different times (0, 1, 3 or 24 hours), an aliquot of cells was taken and stained with FITC-conjugated anti-human IgG and analyzed by flow cytometry. The results obtained at time 0 and 24 hours are shown in FIG. 15A. The mean fluorescence measured at different times was compared to that at time 0 when maximum binding was observed. The time course of antibody binding calculated as % of maximum mean fluorescence is illustrated in FIG. 15B. A gradual decline in antibody cell surface staining intensity was observed with time. Significant staining (42% of staining at time 0) was still detected at 24 hours for both her2.IgG3 and B7.her2.IgG3.

### Example 11

Proliferation Assays

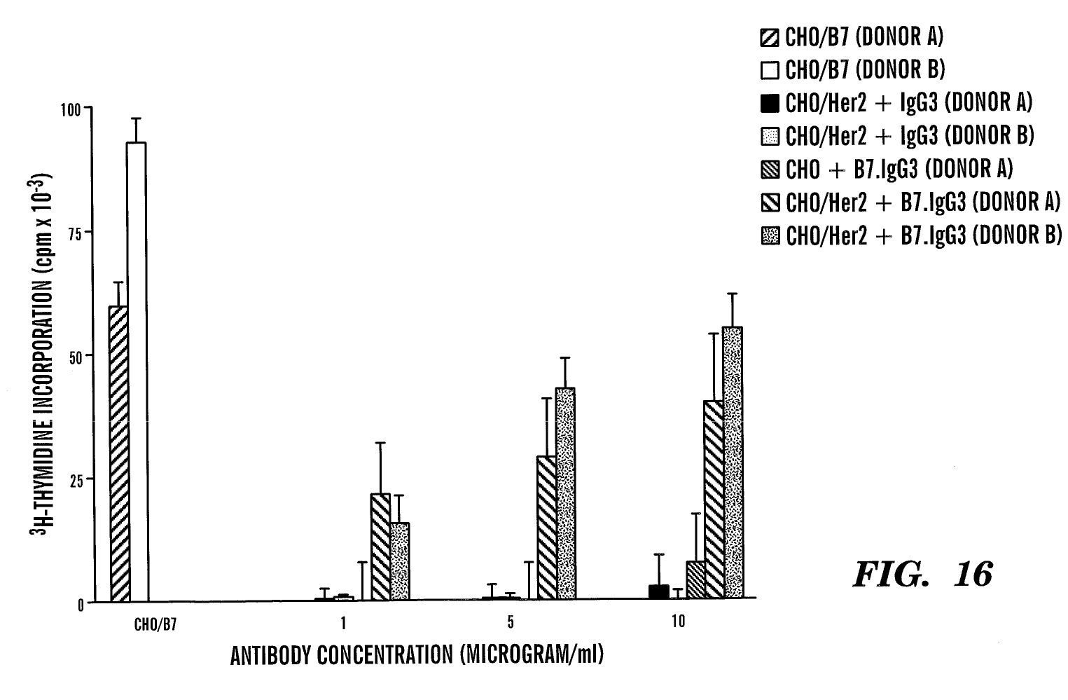

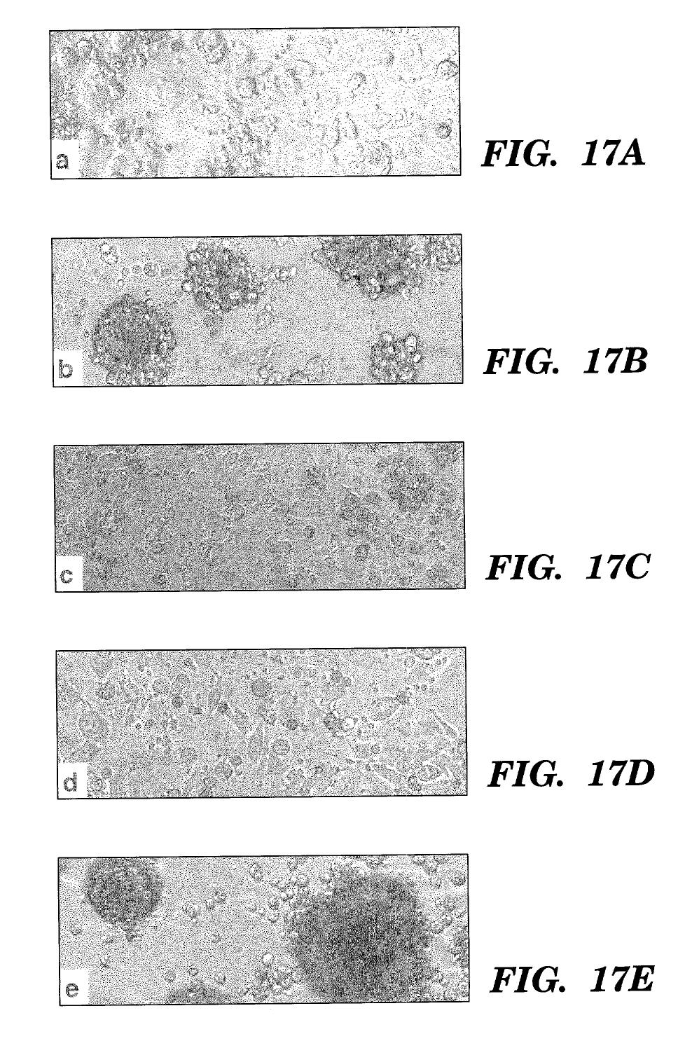

To test for the functional ability of the B7.her2.IgG3 molecule to signal via CD28, a syngeneic T-cell proliferation assay was performed using human peripheral blood T cells (FIG. 16). CHO/Her2 or control CHO cells were irradiated, incubated in presence or absence of either her2.IgG3 or B7.her2.IgG3 and peripheral blood enriched T cells. PMA at 10 ng/ml was added to the cultures to provide signal “one” necessary for proliferation. Addition of B7.her2.IgG3 to CHO/Her2 cells resulted in a dose-dependent increase in T cell proliferation as assayed by ³H-thymidine incorporation. Results from two different donors from two experiments are presented. Levels of T-cell proliferation obtained with 10 μg/ml B7.her2.IgG3 approached the levels obtained through stable expression of human B7.1 in CHO cells by gene transfer (CHO/B7). Proliferation was decreased to absent in presence of parental CHO cells or with control her2.IgG3. The significantly lower levels of proliferation observed when B7.her2.IgG3 was incubated with CHO cells suggests that binding B7.her2.IgG3 to cell surface via the HER2/neu antigen is necessary for optimal T cell costimulation. Visual inspection of the coculture plates showed formation of large foci of proliferating T cells in response to control CHO/B7 cells, or in response to incubation with CHO/Her2 cells in presence of B7.her2.IgG3. Photographs of the cocultures are included in FIG. 17. The presence of proliferating T-cell colonies directly correlated with levels of proliferation detected by ³H-thymidine incorporation.

### Example 12

RANTES and B7.1/anti-her2neu Fusion Proteins

Two additional fusion proteins were constructed in which RANTES is fused at the start of the heavy chain variable domain of anti-her2neu through a flexible linker, and in which B7.1 is fused, through a flexible linker, to the end of the C_{H}3 domain. These fusion proteins contain both RANTES and B7.1 functional domains on the same anti-her2neu antibody molecule. As in the previous examples, the activity of B7.1 and RANTES were determined and both activities were found in the fusion proteins.

Also constructed, was an anti-her2neu IgG3 fusion protein containing either B7.1 or RANTES at the amino terminus of the heavy chain, and the human cytokine interleukin-2 (IL2) joined to the end of the C_{H}3 domain of the heavy chain. These constructs will allow study of the potential synergistic effects of IL2 with either B7.1 or RANTES. The specific activity of IL2 was tested for these chimeric molecules and IL2 was found to retain its activity. B7.1 and RANTES were also shown to maintain their activites. Therfore, these molecules have binding activity specific to a tumor cell antigen, B7.1 or RANTES activity, and IL2 activity.

### Example 13

Animal Models

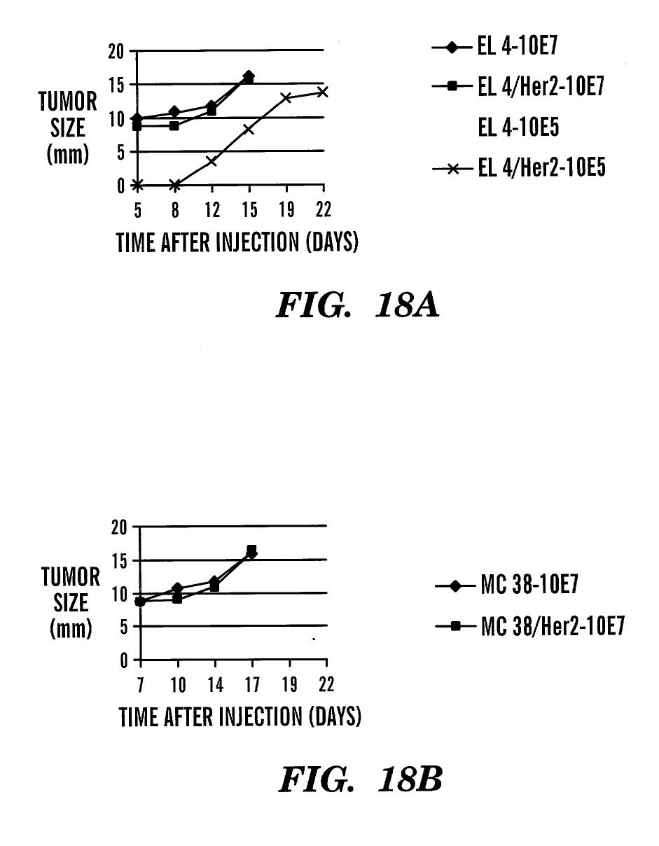

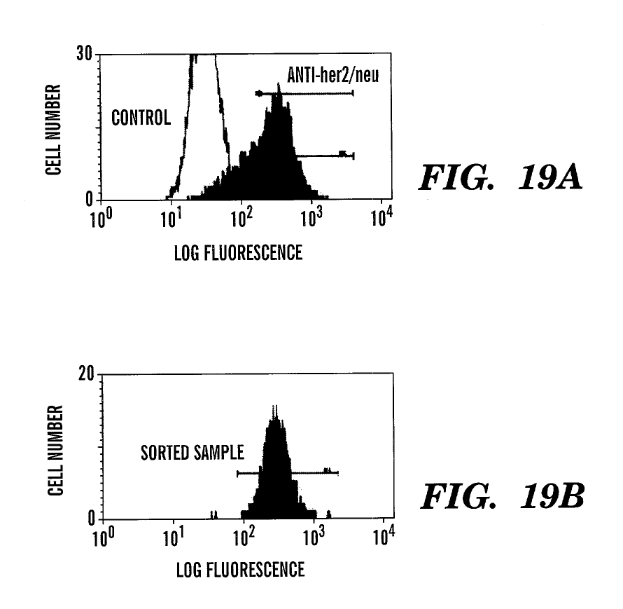

The mouse tumors MC38 and EL4 (derived from the mouse strain C57/B16) were transduced with a retroviral vector expressing the her2/neu CDNA. G418-selected cells were assayed for expression of her2/neu using the 4D5 antibody (obtained from P. Carter, Genentech) by flow cytometry. Bright and dim her2/neu expressing cells were sorted and expanded. MC38 and EL4 cells, sorted for high her2/neu expression, were injected in the flank of C57/BL6 mice. The kinetics of tumor growth of both human-her2neu expressing and parental cells are shown to be similar (FIG. 18). The tumor was dissected from the mouse, dispersed into single cell suspension, and expanded in culture. Persistent her2/neu expression was detected in 75% of the recovered cells (FIG. 19).

As a positive control for later experiments, EL4 and MC38 cell lines stably expressing RANTES and B7.1 were also derived by gene transfer. The cells were sorted for bright expression of B7.1 by flow cytometry, and it was confirmed that the B7.1 transfection into EL4 cells provides protection from tumor growth in vivo when injected in syngeneic mice (Chen, L., et al., “Tumor Immunogenicity Determine the Effect of B7 Costimulation on T Cell-mediated Tumor Immunity,” J Exp Med 179:523 (1994), which is hereby incorporated by reference).

### Example 14

In vivo Pharmacokinetics, Biodistribution, and Imaging

Radiolabeled anti-CEA or her2/neu fusion proteins will be used to determine whether they can detect their antigens on the surface of the transduced cells in vivo, and can be used for in vivo targeting of fusion proteins. The anti-CEA and anti-her2/neu RANTES or B7.1 fusion proteins will be radiolabeled using ¹³¹I by the Pierce Iodobead method (Pierce, Rockford, Ill.). C57BL/6 mice will be injected s.c. in the scapular region with 1×10⁶ MC38/CEA cells, MC-38/her2/neu or control MC-38. Biodistribution studies will be performed, when the tumors will be approximately 0.5 cm in diameter. Mice bearing MC38/CEA tumors will be injected in the tail vein with approximately 3 μCi/mouse¹²⁵I -IgG_{CEA}, and/or ¹³¹I-IgG_{CEA}-B7, and/or ¹³¹I-IgG/RANTES. Anti-her2/neu fusion proteins will be examined in similar fashion in mice bearing her2/neu transduced MC-38 tumors. Mice will be sacrificed at 4 h, 1, 3, 5 and 8 days and blood, tumor and major organs collected, wet-weighed, and radioactivity measured in a multichannel gamma scintillation counter. To verify the tumor localization of fusion proteins, imaging studies will be performed with a gamma camera equipped with a pinhole collimator (a special consideration with respect to the pharmacokinetics and biodistribution of RANTES fusion proteins in the presence of a red blood cell chemokine receptor, reportedly the Duffy antigen) (Neote, K., et al., “Functional and Biochemical Analysis of the Cloned Duffy Antigen: Identity with the Red Blood Cell Chemokine Receptor”, Blood, 84:44 (1994), which is hereby incorporated by reference). Pharmacokinetic studies will need to be performed to ascertain the effect of the red cell “sink” on chemokine fusion protein pharmacokinetics.

### Example 15

Animal Models to Study Effects of Antibody Fusion Molecules on the Immune Response

MC38 cells transfected with human CEA and MC38 and EL4 transduced with her2/neu will be used to study the effects of the antibody fusion molecules on the immune response. Tumorigenicity of the cell lines will be assayed by graded intraperitoneal/flank administration of tumor cells to assay for tumor “take”. Earlier studies by Schlom et al., indicate that a dose of 10⁶ MC38/CEA cells results in detectable flank tumor in all injected mice within 5-7 days (Hand, P. H., et al., “Evaluation of Human CEA-transduced and Non-transduced Murine Tumors as Potential Targets for Anti-CEA Therapies”, Cancer Immunol. Immunother, 36:65 (1993); Robbins, P. F., et al., “Transduction and Expression of the Human Carcinoembryonic Antigen Gene in a Murine Colon Carcinoma Cell Line,” Cancer Res. 51:3657 (1991), which are hereby incorporated by reference). Additional studies have confirmed these observations. Preliminary studies also indicate adequate growth of both EL4 and MC38 transduced with her2/neu. Once Minimal Tumorigenic Doses (MiTD) have been ascertained for both parental and transduced tumors, biolocalization and challenge experiments will be performed as outlined above.

Specific animal models may need to be developed, as not all mouse tumors are rendered immunogenic by B7.1 transduction. If this is the case for MC38, the EL-4 (T-lymphoma) for which B7.1 transduction has demonstrated protective effects will be used (Chen, L., et al, “Tumor Immunogenicity Determine the Effect of B7 Costimulation on T Cell-mediated Tumor Immunity,” J Exp Med, 179:523 (1994), which is hereby incorporated by reference). Additional cell lines transduced with retroviral vectors which express RANTES or B7.1 have been established and characterized for comparison.

Initial challenge experiments will be compared using graded tumor doses coated ex vivo with antibody controls or antibody fusion proteins. Animals which reject doses equal to/exceeding the MiTD will be rechallenged with parental tumor (non-transduced). Spleens from protected, and non-protected animals will be harvested and cells characterized for CTL activity by ⁵¹Cr release assays. Whether systemic administration of antibody fusion proteins will confer a protective effect against parental cells will be determined. Animals challenged with MiTD of tumor cells expressing relevant tumor antigens, will be inoculated with tumor, followed one or more days later by intraperitoneal injection of fusion proteins or control antibodies in escalating doses and studied as above.

The examples demonstrate the construction and characterization of an antibody-chemokine fusion protein in which the chemokine RANTES was linked by genetic engineering to an antibody specific for the tumor associated antigen HER2/neu. RANTES.Her2.IgG3 should localize to the tumor vicinity through the antibody domain of the fusion protein. The accumulation of the fusion protein at the tumor site should then create a local chemokine gradient which would enhance the transendothelial migration of effector cells such as T-lymphocytes, natural killer cells, monocytes and dendritic cells. The increase in immune effectors could then enhance the development of an active cellular immune response at the site of the tumor.

The anti-HER2/neu antibody used in this study is based on the humanized humAb4D5-8 antibody current in Phase III clinical trials (Carter, P., et al., “Humanization of an Anti-p185HER2 Antibody for Human Cancer Therapy”, Proc Natl Acad Sci USA, 89:4285-9 (1992); Baselga, J., et al., “Phase II Study of Weekly Intravenous Recombinant Humanized Anti-185HER2 Monoclonal Antibody in Patients with HEr2/neu-overexpressing Metastatic Breast Cancer [see comments],” J Clin Oncol, 14:737-44 (1996), which are hereby incorporated by reference). The variable sequences of the antibody were cloned into a human IgG3 backbone in order to provide greater flexibility in folding of the fusion protein mediated by the long hinge region of IgG3. The examples indicate that RANTES can be effectively linked to the amino terminus of the heavy chain of the antibody, with retention of both antibody specificity and RANTES activity. The examples also demonstrate that anti-HER2/neu affinity of the RANTES.Her2.IgG3 fusion protein for its antigenic target is similar to that of the IgG3 parental antibody. In an assay of biological activity, RANTES.Her2.IgG3 was capable of inducing F-actin polymerization of monocytic cells. It was consistently observed that the activity of RANTES in the fusion protein is higher than rRANTES on a molar basis. This may be due to the fact that the larger molecular weight of RANTES.Her2.IgG3 fusion protein (185 kDA versus 8 kDa for rRANTES) is providing greater stability of the fusion protein and thereby greater activity. Alternatively, the bivalency of RANTES in RANTES.Her2.IgG3 may increase its potency. In assays for transendothelial migration in vitro, both peripheral blood T cells and monocytes were shown to migrate in response to RANTES.Her2.IgG3 fusion protein, while limited migration was observed using the IgG3 antibody. This suggests that the antibody fusion protein in soluble form is capable of effectively stimulating transendothelial migration of inflammatory cells.

The chemotactic effect of chemokines appears to be mediated by the generation of a chemokine gradient in the tumor vicinity. To test for the ability of RANTES antibody fusion protein to elicit a gradient when bound to antigen-expressing cells, the effect of cell-surface chemotactic effects exhibited by cell-surface immobilized RANTES.Her2.IgG3 was measured. Anti-HER2/neu RANTES.Her2.IgG3 bound to SKBR3 cells was capable of inducing transendothelial migration of T cells in a transwell migration assay. Antibody affinity, avidity, as well as equilibrium binding (association and dissociation) may all contribute to the generation of a local RANTES gradient by the fusion protein. Shedding of the HER2/neu antigen-fusion protein complex may also contribute to the formation of a gradient. Such shedding of HER2/neu antigen along, or following binding of antibody has been observed in vitro, and soluble HER2/neu (ECD) can be measured in vivo in breast cancer patients (Pupa, S. M., et al., “The Extracellular Domain of the c-erbB-2 Oncoprotein is Released From Tumor Cells by Proteolytic Cleavage,” Oncoqene, 8:2917-23 (1993), which is hereby incorporated by reference). A member of a new class of chemokines, a CX₃C chemokine, expressed by endothelial cells has been recently described (Bazan, J. F., et al., “A New Class of Membrane-bound Chemokine with a CX3C Motif,” Nature, 385:640-4 (1997), which is hereby incorporated by reference). The CX₃C molecule exists in a secreted and membrane bound form as further evidence that a membrane bound chemokine, if also present in soluble form at enough level, can promote effector cell migration.

RANTES has been reported to induce two calcium influx signals in T cells. The first, is of short duration and characteristic of chemokines, whereas the second is similar to the T cell receptor activation signal leading to antigen-independent T cell proliferation (Bacon, K., et al., “Activation of Dual T Cell Signaling Pathways by the Chemokine RANTES,” Science, 269:1727-1730 (1995), which is hereby incorporated by reference). Taub et al (1996) have shown that RANTES can also potentiate B7.1-mediated T cell costimulation (Taub, D., et al., “Chemokines and T Lymphocyte Activation: I. Beta Chemokines Costimulate Human T Lymphocyte Activation in Vitro,” J Immunol, 156:2095-2103 (1996), which is hereby incorporated by reference). Synergy may exist between the RANTES.Her2.IgG3 fusion protein and another fusion protein in which the extracellular domain of B7.1 costimulatory molecule was fused to an antitumor antibody (Challita-Eid P., Penichet M. L., Shin S. U., Poles T. M., Mosammaparast, N., Mahmood K., Slamon D. J., Morrison S. L., Rosenblatt J. D., manuscript submitted). RANTES was recently shown to generate an antitumor immune response when MCA-205 sarcoma cells engineered to express RANTES were injected in vivo in syngeneic immunocompetent mice (Mule, J., et al., “RANTES Secretion By Gene-Modified tumor Cells Results in Loss of Tumorigenicity In Vivo: Role of Immune Cell Subpopulations,” Hum Gene Ther, 7:1545-1553 (1996), which is hereby incorporated by reference). Similar results were seen using the murine EL4 lymphoma. RANTES was observed to provide protection from tumor grow whether introduced stably ex vivo through retroviral vectors, or introduced transiently through herpes-derived amplicon vector in vivo in established tumors (Mahmood K., Federoff H., Haltman M., Challita-Eid P. m., Rosenblatt, J. D., manuscript submitted). Protection is associated with an increase in cytotoxic T lymphocyte activity and development of systemic immunity capable of rejecting parental non-RANTES expressing tumor cells upon rechallenge. Therefore, local delivery of RANTES may be a suitable strategy for the recruitment and activation of a tumor specific immune response.

One potential limitation of the bioavailability of RANTES-antibody fusion protein is the presence of a promiscuous receptor for C-C and C-X-C chemokines on the surface of red blood cells which may serve as a “sink” for free chemokines (Horuk, R., et al., “Identification and Characterization of a Promiscuous Chemokine-binding Protein in a Human Erythroleukemic Cell Line,” J Biol Chem, 269:17730-3 (1994), which is hereby incorporated by reference). While chemokine receptor/ligand interactions on target inflammatory cells appear to be specifically regulated, erythrocytes have been observed to possess a multispecific receptor which binds chemokines of both C-C and C-X-C classes. This receptor has been cloned and shown to be identical to the “Duffy” antigen (Horuk, R., et al., “Identification and Characterization of a Promiscuous Chemokine-binding Protein in a Human Erythroleukemic Cell Line,” J Biol Chem, 269:17730-3 (1994); Lu, Z. H., et al., “The Promiscuous Chemokine Binding Profile of the Duffy Antigen/Receptor for Chemokines is Primarily Localized to Sequences in the Amino-Terminal Domain,” J Biol Chem, 270:26239-26245 (1995), which are hereby incorporated by reference). The effects of red cell binding on RANTES antibody fusion protein activity are currently being investigated. It is now known whether fusion decreases the affinity of RANTES for the erythrocyte chemokine receptor. It also may be possible to mutate RANTES so that it no longer binds the RBC receptor but retains its ability to recruit immune effector cells. RANTES was chosen initially in these studies because of its dual function as a chemoattractant and a stimulant of T cell activation. However, other chemokines which do not bind to the Duffy antigen, may also be suitable candidates for fusion with an antibody. Alternatively, RANTES.Her2.IgG3 fusion protein could be delivered intratumorally or in settings in which red cell binding is less likely to present a problem, such as for intraperitoneal or intrapleural disease.

The examples also show the construction and characterization of a fusion antibody in which the extracellular domain of the B7.1 costimulatory molecule was fused by genetic engineering to the amino terminus of the heavy chain of an anti-HER2/neu antibody. The IgG3 backbone was chosen for the antibody molecule since the extended hinge region of IgG3 would be expected to provide greater flexibility in folding to accommodate the presence of B7.1 in the fusion antibody. IgG3 also exhibits Fc mediated functions such as complement activation and Fcγ binding (Morrison, S., In Vitro Antibodies: “Strategies for Production and Application,” Annu Rev Immunol 10:239-65 (1992), which is hereby incorporated by reference). The B7.1 costimulatory ligand was chosen in preference to B7.2, as Gajewski et al. and other investigators have suggested that B7.1 transduced tumors more successfully induce CTL activity, and protect against parental tumor challenge more effectively than tumors transduced with B7.2 (Matulonis, U. et al., “B7-1 is Superior to B7-2 Costimulation in the Induction and Maintenance of T Cell-Mediated Antileukemia Immunity. Further Evidence that B7-1 and B7-2 are Functionally Distinct,” J Immunol 156:1126-31 (1996); Gajewski, T., et al., “Tumor Rejection Requires a CTLA4 Ligand Provided by the Host or Expressed on the Tumor: Superiority of B7-1 over B7-2 for Active Tumor Immunization,” J Immunol 156:2909-17 (1996); Gajewski, T., “B7-1 but not B7-2 Efficiently Costimulates CD8+ T Lymphocytes in the P815 Tumor System in Vitro,” J Immunol 156:465-72 (1996); Chamberlain, R. et al., “Costimulation Enhances the Active Immunotherapy Effect of Recombinant Anticancer Vaccines,” Cancer Res 56:2832-6 (1996), which are hereby incorporated by reference). Although conflicting results with respect to Th1 versus Th2 differentiation have been reported using B7.1 and B7.2, results from several experimental systems suggest that B7.1 costimulation tends to favor differentiation along the Th1 pathway (Guinan, E. et al., “Pivotal Role of the B7:CD28 Pathway in Transplantation Tolerance and Tumor Immunity,” Blood 84:3261-82 (1994); Freeman, G., et al., “B7-1 and B7-2 do not Deliver Identical Costimulatory Signals, Since B7-2 but not B7-1 Preferentially Costimulates the Initial Production of IL-4,” Immunity 2:523-532 (1995); Greenfield, E. et al., “B7.2 Expressed by T Cells does not Include CD28-Mediated Costimulatory Activity but Retains CTLA4 Binding: Implications for Induction of Antitumor Immunity to T Cell Tumors,” J Immunol 158:2025-34 (1997); Kuchroo, V. et al., “B7-1 and B7-2 Costimulatory Molecules Activate Differentially the Th1/Th2 Developmental Pathways: Application to Autoimmune Disease Therapy,” Cell 80:707-18 (1995), which are hereby incorporated by reference). Therefore B7.1, rather than B7.2, was linked to an antitumor antibody in an effort to preferentially stimulate a Th1 mediated immune response.

The results indicate that B7.1 can be effectively linked to the amino terminus of the heavy chain of an anti-HER2/neu antibody, with retention of both antibody specificity and the B7.1 interaction with CD28. Binding to HER2/neu was demonstrated by flow cytometry, as well as IAsys biosensor studies, albeit at a lower affinity than that observed for the control her2.IgG3. Possible reasons for the observed decrease in affinity could be steric hindrance between the anti-HER2/neu variable and the B7.1 domains, or a change in the kinetics of antigen binding due to the increased size of B7.her2.IgG3. Similarly, specificity of B7.1 for both CTLA4 and CD28 was demonstrated by the ability of B7.her2.IgG3 to bind soluble CTLA4Ig and CD28Ig, as well as CD28 expressed on the surface of target cells. Of note, in preliminary attempts to derive a fusion antibody, an anti-dansyl antibody fusion was constructed in which the B7.1 coding sequences were fused to the carboxyl end of the heavy chain C_{H}3 domain. No binding of this B7.1 fusion antibody to CD28 expressed on the surface of CHO/CD28 cells was observed, nor to soluble CD28Ig, suggesting that fusion through the B7.1 amino terminus may disrupt the B7.1/CD28 interaction. This may be due to masking of the amino terminus sequences of B7.1, known to be in close proximity to the CD28/CTLA4 binding site (Guo, Y., et al., “Mutational Analysis and an Alternatively Spliced Product of B7 Defines its CD28/CTLA4-Binding Site on Immunoglobulin C-Like Domain,” J Exp Med 181:1345-1355 (1995), which is hereby incorporated by reference). Whether fusion via a flexible linker will restore binding of B7.1 to CD28 is currently under investigation in the laboratory. At present, however, fusion of B7.1 sequences to the amino terminal heavy chain sequences is preferred for suitable B7.1/CD28 interaction. A similar requirement for fusion at the amino terminus of the antibody to maintain activity was observed for nerve growth factor (McGrath, J. et al., “Bifunctional Fusion Between Nerve Growth Factor and a Transferrin Receptor Antibody,” J Neurosci Res 47:123-33 (1997), which is hereby incorporated by reference). Since the Fc domain remains intact in B7.her2.IgG3, binding to Fc receptors may induce ADCC or otherwise affect B7.1 function. If this is a problem, further manipulation of the constant region could be performed to eliminate FcR binding sites.

Anti-HER2/neu antibodies were also examined to determine whether they would remain on the surface of antigen presenting breast cancer cells. The results indicated that approximately 40% of surface bound B7.her2.IgG3 was detectable by flow cytometry for up to 24 hours following initial incubation with human SKBR3 breast cancer cells. This suggests that stable presentation of the B7.1 costimulatory ligand on the tumor cell surface may be feasible, and that loss of presentation due to internalization or rapid antigenic shedding via HER2/neu binding may not be a significant problem (Tagliabue, E. et al., “Selection of Monoclonal Antibodies Which Induce Internalization and Phosphorylation of p185HER2 and Growth Inhibition of Cells with HER2/NEU Gene Amplification,” Int J Cancer 47:933-7 (1991), which is hereby incorporated by reference).

The management of minimal residual disease is a central problem in breast cancer and other solid tumors. Despite the use of increased the dose intensity of chemotherapy or autologous bone marrow transplantation, relapse remains a critical problem (Harris, L. et al., “The Role of Primary Chemotherapy in Early Breast Cancer,” Semin Oncol 23:31-42 (1996); Sledge, G., “Adjuvant Therapy for Early Stage Breast Cancer,” Semin Oncol 23:51-4 (1996); Seidman, A., “Chemotherapy for Advanced Breast Cancer: A Current Perspective,” Semin Oncol 23:55-9 (1996); Bearman, S. et al., “High-Dose Chemotherapy with Autologous Hematopoietic Progenitor Cell Support for Metastatic and High-Risk Primary Breast Cancer,” Semin Oncol 23:60-7 (1996), which are hereby incorporated by reference). Chemotherapeutic strategies are necessarily limited by various toxicities. Additional modalities, which can achieve further cytoreduction are needed. Although various clinical trials of monoclonal antibodies, antibody based conjugates and/or radioantibodies have been performed, results of these trials have highlighted obstacles to successful antibody-based therapy of human malignancy. Antibodies generally are not directly cytotoxic, due to poor fixation of complement and/or inadequate activation of antibody dependent cytotoxicity. Effective use of antibodies for delivering cytotoxic agents (e.g. conjugates such as antibody-ricin, or radiolabeled antibody strategies) requires delivery to a majority of, if not all tumor cells (Rodrigues, M. et al., “Development of a Humanized Disulfide-Stabilized Anti-p185HER2 Fv-beta-lactamase Fusion Protein for Activation of a Cephalosporin Doxorubicin Prodrug,” Cancer Res 55:63-70 (1995), which is hereby incorporated by reference). An alternative approach is to elicit an active systemic immune response against tumor cells. Delivery of cytokines has been shown to induce an antitumor T-cell response. Although gene transfer has most commonly been used to achieve increased cytokine levels at the site of the tumor, recent studies performed using an antibody-IL2 fusion protein suggests that antibodies can be used for delivering cytokines to tumors. The antibody-cytokine fusion protein retains both antibody specificity and cytokine activity and appears to be more effective than either used alone or in combination, but not covalently linked (Becker, J. et al., “Long-Lived and Transferable Tumor Immunity in Mice After Targeted Interleukin-2 Therapy,” J Clin Invest 98:2801-4 (1996); Becker, J. et al., “T Cell-Mediated Eradication of Murine Metastatic Melanoma Induced by Targeted Interleukin 2 Therapy,” J Exp Med 183:2361-6 (1996); Becker, J. et al., “An Antibody-Interleukin 2 Fusion Protein Overcomes Tumor Heterogeneity by Induction of a Cellular Immune Response,” Proc Natl Acad Sci USA 93:7826-31 (1996); Sabzevari, H. et al., “A Recombinant Antibody-Interleukin 2 Fusion Protein Suppresses Growth of Hepatic Human Neuroblastoma Metastases in Severe Combined Immunodeficiency Mice,” Proc Natl Acad Sci USA 91:9626-30 (1994); Harvill, E. et al., “In Vivo Properties of an IgG3-IL-2 Fusion Protein. A General Strategy for Immune Potentiation,” J Immunol 157:3165-70 (1996); Harvill, E. et al., “An IgG3-IL-2 Fusion Protein has Higher Affinity than hrIl-2 for the IL-2R Alpha Subunit: Real Time Measurement of Ligand Binding,” Mol Immunol 33:1007-14 (1996), which are hereby incorporated by reference). However, fusion of B7.1 rather than cytokine should result in activation of T-cells with TCRs which specifically recognize tumor determinants rather than the nonspecific activation expected of a fused cytokine.

In assays for T-cell costimulation in vitro, effective stimulation of human T-cells was achieved only if B7.her2.IgG3 was bound to a HER2/neu target, and limited or no stimulation was observed using target cells which did not express HER2/neu antigen. This suggests that B7.her2.IgG3 fusion protein in soluble form may not be able to effectively provide a costimulatory signal to preactivated T-cells, and that anti-HER2/neu antibody domain in the B7.her2.IgG3 fusion protein provided specificity for the T-cell costimulation. This property of B7.her2.IgG3 would allow enhance specificity of the immune response. Similar results have been reported using fusion of the B7.2 costimulatory ligand to a single chain antibody (Gerstmayer, B. et al., “Costimulation of T Cell Proliferation by a Chimeric B7-2 Antibody Fusion Protein Specifically Targeted to Cells Expressing the erbB2 Proto-Oncogene,” J Immunol 158:4584-90 (1997), which is hereby incorporated by reference). However, a single chain antibody, produced in yeast cells may have considerably different glycosylation and antigenicity as well as different pharmacokinetics in vivo compared to humanized B7.her2.IgG3. The relative specificity and type of response achieved with B7.her2.IgG3 fusion compared to the B7.2 single chain fusion remains to be determined. It also remains to be determined whether the specificity and response achieved with B7.1 fusions will differ from that observed using bispecific antitumor/anti-CD28 antibody (Guo, Y. et al., “Effective Tumor Vaccines Generated by in Vitro Modification of Tumor Cells with Cytokines and Bispecific Monoclonal Antibodies,” Nat Med 3:451-5 (1997), which is hereby incorporated by reference). However, genetically-engineered antibody fusion proteins should present fewer problems in manufacture and purification than the described bispecific antibodies, which are difficult to purify to homogeneity.

In conclusion, the examples show the construction and characterization of a chemokine antibody fusion protein with specificity for a tumor associated antigen. While several antibody cytokine fusion proteins have been described (Becker, J. C., et al., “Long-lived and Transferable Tumor Immunity in Mice after Targeted Interleukin-2 Therapy,” J Clin Invest, 98:2801-4 (1996); Becker, J. C., et al., “T Cell-Mediated Eradication of Murine Metastatic melanoma Induced by Targeted Interleukin 2 Therapy,” J Exp Med, 183:2361-6 (1996); Harvill, E. T., et al., “An IgG3-IL-2 Fusion Protein Has Higher Affinity Than hrIL-2 for the IL-2R Alpha Subunit: Real Time Measurement of Ligand Binding,” Mol Immunol, 33:1007-14 (1996); Harvill, E. T., et al., “In Vivo Properties of an IgG3-IL-2 Fusion Protein. A General Strategy For Immune Potentiation,” J Immunol, 157:3165-70 (1996); Syrengelas, A. D., et al., “DNA Immunization Induces Protective Immunity Against B-cell Lymphoma,” Nat Med, 2:1038-1041 (1996) which are hereby incorporated by reference), this is the first report of an antibody chemokine fusion protein. Such a fusion protein has the potential to recruit a large repertoire of T cells and other inflammatory cells to the tumor vicinity and thereby enhance the antitumor immune response. Recruitment of a large cohort of effector cells may augment the likelihood of activating tumor specific memory cells or may allow activation of naive T cells through exposure to additional costimulatory signals as well as processed tumor antigens. Chemokine-antibody fusion proteins might be useful, alone or in combination with other previously described fusion proteins such as fusions with IL2 (Harvill E. T. et al., “An IgG3-IL-2 Fusion Protein Has Higher Affinity Than hrIL-2 for the IL-2R Alpha Subunit: Real Time Measurement of Ligand Binding,” Mol Immunol, 33:1007-14 (1996); Harvill, E. T. et al., “In Vivo Properties of an IgG3-IL-2 Fusion Protein. A General Strategy for Immune Potentiation,” J Immunol, 157:3165-70 (1996), which are hereby incorporated by reference) and/or B7.1 (Challita-Eid P., Penichet M. L., Shin S. U., Poles, T. M., Mosammaparast N., Mahmood K., Slamon D. J., Morrison S. L., Rosenblatt J. D., manuscript submitted) in eliciting an enhanced immune response to tumors.

The examples also show the production of an anti-HER2/neu IgG fusion protein encoding the extracellular domain of the B7.1 costimulatory ligand. This protein retains targeting specificity via the HER2/neu antigen, as well as ability to deliver a T-cell costimulatory signal. The strategy offers several theoretical advantages. While expression of HER2/neu may be heterogenous, targeting via HER2/neu may activate T-cells with specificity against other unidentified tumor associated antigens, resulting in destruction of both HER2/neu positive and nonexpressing cells. Therefore, the antibody fusion protein may allow targeting of micrometastatic disease with relative specificity and would not itself have to bind to all tumor cells to elicit an effective response. The data presented suggest that tumor specific antibodies fused with costimulatory ligands may be a useful method for delivering a costimulatory signal for the purpose of cancer immunotherapy.

Although preferred embodiments have been depicted and described in detail herein, it will be apparent to those skilled in the relevant art that various modifications, additions, substitutions, and the like can be made without departing from the spirit of the invention and these therefore are considered within the scope of the invention as defined in the claims which follow.

What is claimed:

1. A chimeric molecule suitable for stimulating a tumor specific immune response comprising:
a binding domain capable of specifically binding to a tumor cell associated antigen, and
a chemokine or active fragment thereof, which is associated with the binding domain such that the binding domain remains capable of binding to the tumor cell associated antigen and the chemokine retains activity.

2. The chimeric molecule according to claim 1, wherein the binding domain is an antibody or fragment thereof which specifically binds to the tumor associated antigen.

3. The chimeric molecule according to claim 2, wherein the chemokine or active fragment thereof is linked to the amino terminus of the heavy or light chain of the antibody.

4. The chimeric molecule according to claim 3, wherein the chemokin or active fragment thereof is linked to the amino terminus of the heavy chain of the antibody.

5. The chimeric molecule according to claim 1, further comprising:
a flexible linker or hinge region connecting the chemokine and the binding domain.

6. The chimeric molecule according to claim 1, wherein the chemokine is selected from the group consisting of DC-CK1, SDF-1, fractalkine, lymphotactin, IP-10, Mig, MCAF, MIP-1α, MIP-1β, IL-8, NAP-2, PF-4, and RANTES or an active fragment thereof.

7. The chimeric molecule according to claim 6, wherein the chemokine is RANTES.

8. The chimeric molecule according to claim 1, wherein the binding domain specifically binds to a tumor cell associated antigen from tumor cells selected from the group consisting of breast cancer cells, ovarian cancer cells, lung cancer cells, bladder caner cells, and prostate cancer cells.

9. The chimeric molecule according to claim 1, wherein the binding domain specifically binds to her2/neu.

10. The chimeric molecule according to claim 1, wherein the tumor cell associated antigen is a cell surface antigen.

11. A gene encoding the chimeric molecule of claim 1.

12. The gene according to claim 11, wherein the gene is functionally linked to a promoter.

13. An expression vector carrying the gene of claim 10.

14. The expression vector according to claim 13, wherein the vector is a viral vector, plasmid, cosmid, or an oligonucleotide.

15. A host cell transduced with the gene of claim 11.

16. A method for stimulating a tumor specific immune response comprising:
contacting the tumor cells in a mammal with the chimeric molecule according to claim 1 under conditions effective to stimulate an immune response.

17. The method for stimulating a tumor specific immune response according to claim 16, wherein said contacting comprises:
administering the chimeric molecule to a mammal.

18. The method for stimulating a tumor specific immune response according to claim 16, wherein said contacting comprises:
introducing a gene capable of expressing the chimeric moleucule into cells of a mammal, and
expressing the chimeric molecule from the gene.

19. The method according to claim 16, wherein the chemokine is selected from the group consisting of DC-CK1, SDF-1, fractalkine, lymphotactin, IP-10, Mig, MCAF, MIP-1α, MIP-1β, IL-8, NAP-2, PF-4 , and RANTES or an active fragment thereof.

20. The chimeric molecules according to claim 19, wherein the chemokine is RANTES.

21. The method according to claim 16, wherein the binding domain specifically binds tumor cell associated antigen from tumor cells selected from the group consisting of breast cancer cells, ovarian cancer cells, lung cancer cells, bladder cancer cells, and prostate cancer cells.

22. The method according to claim 16, wherein the binding domain specifically binds to her2/neu.

23. The method according to claim 16, wherein the binding domain specifically binds to a cell surface antigen.

24. The method according to claim 16, wherein said administering is oral, in dermal, intramuscular, interperitoneal, intravenous, subcutaneous, or intranasal.

25. A composition for stimulating a tumor specific immune response comprising:
the chimeric molecule according to claim 1, and
a pharmaceutically-acceptable carrier.

26. A chimeric molecule suitable for stimulating a tumor specific immune response, comprising:
a binding domain capable of binding to a tumor cell associated antigen, and
a costimulatory ligand or active fragment thereof, which is associated with the binding domain such that the binding domain remains capable of binding to the tumor cell associated antigen and the costimulatory ligand retains activity.

27. The chimeric molecule according to claim 26, wherein the binding domain is an antibody or fragment thereof which specifically binds to the tumor associated antigen.

28. The chimeric molecule according to claim 27, wherein the costimulatory ligand or active fragment thereby is linked to the amino terminus of the heavy or light chain of the antibody.

29. The chimeric molecule according to claim 28, wherein the costimulatory ligand or active fragment thereof is linked to the amino terminus of the heavy chain of the antibody.

30. The chimeric molecule according to claim 26, further comprising:
a flexible linker or hinge region connecting the costimulatory ligand and the binding domain.

31. The chimeric molecule according to claim 26, wherein the costimulator ligand is B7.1 or B7.2.

32. The chimer molecule according to claim 31, wherein the costimulatory ligand is B7.1.

33. The chimeric molecule according to claim 26, wherein the binding domain specifically binds tumor cell associated antigen from tumor cells selected from the group consisting of breast cancer cells, ovarian cancer cells, lung cancer cells, bladder cancer cells, and prostate cancer cells.

34. The chimeric molecule according to claim 26, wherein the binding domain specifically binds to her2/neu.

35. The chimeric molecule according to claim 26, wherein the tumor cell associated antigen is a cell surface antigen.

36. A gene encoding the chimeric molecule of claim 26.

37. The gene according to claim 36, wherein the gene is functionally linked to a promoter.

38. An expression vector carrying the gene of claim 37.

39. The expression vector according to claim 38 wherein the vector is viral vector, plasmid, cosmid, or an oligonucleotide.

40. A host cell transduced with the gene of claim 36.

41. A method for stimulating a tumor specific immune response comprising:
contacting the tumor cells in a mammal with the chimeric molecule according to claim 26 under conditions effective to stimulate an immune response.

42. The method for stimulating a tumor specific immune response according to claim 41, wherein said contacting comprises:
administering the chimeric molecule to a mammal.

43. The method for stimulating a tumor specific immune response according to claim 41, wherein said contacting comprises:
introducing a gene capable of expressing the chimeric moleucule into cells of a mammal, and
expressing the chimeric molecule from the gene.

44. The method according to claim 41, wherein the costimulatory ligand is B7.1 or B7.2.

45. The method according to claim 44, wherein the costimulatory ligand is B7.1.

46. The method according to claim 41, wherein the binding domain specifically binds tumor cell associated antigen from tumor cells selected from the group consisting of breast cancer cells, ovarian cancer cells, lung cancer cells, bladder cancer cells, and prostate cancer cells.

47. The method according to claim 41, wherein the binding domain specifically binds to her2/neu.

48. The method according to claim 41, wherein the tumor cell associated antigen is a cell surface antigen.

49. The method according to claim 41, wherein said administering is oral, subcutaneous, intradermal, intramuscular, intraperitoneal, intrapleural, intravenous, or intranasal.

50. A composition or stimulating a tumor specific immune response comprising:
the chimeric molecule according to claim 26, and
a pharmaceutically-acceptable carrier.

51. A method for stimulating a tumor specific immune response comprising:
contacting the tumor cells in a mammal with the a first chimeric molecule comprising a binding domain capable of binding to a tumor cell associated antigen and a chemokine or active fragment thereof and a second chimeric molecule comprising a binding domain capable of binding to a tumor cell associated antigen and a cost mulatory ligand or active fragment thereof under conditions effective to stimulate an immune response.

52. The method for stimulating a tumor specific immune response according to claim 51, wherein said contacting comprises:
administering the chimeric molecules to a mammal.

53. The method for stimulating a tumor specific immune response according to claim 51, wherein said contacting comprises:
introducing genes capable of expressing the chimeric moleucules into cells of a mammal, and
expressing the chimeric molecules from the genes.

54. The method according to claim 51, wherein the chemokine is selected from the group consisting of DC-CK1, SDF-1, fractalkine, lymphotactin, IP-10, Mig, MCAF, MIP-1α, MIP-1β, IL-8, NAP-2, PF-4, and RANTES or an active fragment thereof.

55. The method according to claim 54, wherein the chemokine is RANTES.

56. The method according to claim 51, wherein the costimulatory ligand is B7.1 or B7.2.

57. The method according to claim 56, wherein the costimulatory ligand is B7.1.

58. The method according to claim 51, wherein each or both binding domains specifically bind tumor cell associated antigen from tumor cells selected from the group consisting of breast cancer cells, ovarian cancer cells, lung cancer cells, bladder cancer cells, and prostate cancer cells.

59. The method according to claim 51, wherein each or both binding domains specifically bind to her2/neu.

60. The method according to claim 51, wherein each or both binding domains specifically bind to a cell surface antigen.

61. The method according to claim 51, wherein said administering is oral, intradermal, intramuscular, intraperitoneal, intrapleural, intravenous, subcutaneous, or intranasal.

62. A composition for stimulating a tumor specific immune response comprising:
a chimeric molecule comprising a binding domain capable of binding to a tumor cell associated antigen and a chemokine or active fragment thereof;
a chimeric molecule comprising a binding domain capable of binding to a tumor cell associated antigen and a costimulatory ligand or active fragment thereof; and
a pharmaceutically-acceptable carrier.

63. A chimeric molecule suitable for stimulating a tumor specific immune response, comprising:
a binding domain capable of specifically binding to a tumor cell associated antigen, and
two or more T-cell effectors selected from the group comprising a chemokine or active fragment thereof, a cytokine or active fragment thereof, and a costimulatory molecule or active fragment thereof, which are associated with the binding domain such that the binding domain remains capable of binding the tumor cell associated antigen and the T-cell effectors retain activity.

64. The chimeric molecule of claim 63, wherein the T-cell effectors are a chemokine and a costimulatory molecule.

65. The chimeric molecule of claim 64, wherein the chemokine is RANTES.

66. The chimeric molecule of claim 64, wherein the costimulatory molecule is B7.1.

67. The chimeric molecule of claim 63, wherein the T-cell effectors are a chemokine and a cytokine.

68. The chimeric molecule of claim 67, wherein the chemokine is RANTES.

69. The chimeric molecule of claim 67, wherein the cytokine is IL-2.

70. The chimeric molecule of claim 63, wherein the T-cell effectors are a cytokine and a costimulatory molecule.

71. The chimeric molecule of claim 70, wherein the cytokine is IL-2.

72. The chimeric molecule of claim 70, wherein the costimulatory molecule is B7.1.

73. The chimeric molecule of claim 63, wherein the T-cell effectors are a chemokine, a cytokine and a costimulatory molecule.

74. The chimeric molecule of claim 73, wherein the chemokine is RANTES.

75. The chimeric molecule of claim 73, wherein the cytokine is IL-2.

76. The chimeric molecule of claim 73, wherein the costimulatory molecule B7.1.
